(19) United States
(12) Patent Application Publication
(10) Pub. No.: US 2010/0210694 A1
(43) Pub. Date: Aug. 19, 2010

(54) NEW NO-DONOR ASPIRIN DERIVATIVES

(75) Inventors: Roberta Fruttero, Savigliano (Cuneo) (IT); Alberto Gasco, Moncalieri (Torino) (IT); Loretta Lazzarato, Rivoli (Torino) (IT); Monica Donnola, Torino (IT); Barbara Rolando, Giaveno (Torino) (IT); Stefano Biondi, Pero (Milano) (IT)

Correspondence Address: ARENT FOX LLP, 1050 CONNECTICUT AVENUE, N.W., SUITE 400, WASHINGTON, DC, 20036, US

(73) Assignee: NICOX S.A., Sophia Antipolis-Valbonne (FR)

(21) Appl. No.: 12/679,594

(22) PCT Filed: Sep. 3, 2008
(86) PCT No.: PCT/EP08/61596

§ 371 Date: Mar. 23, 2010

Related U.S. Application Data
(60) Provisional application No. 60/960,912, filed on Oct. 19, 2007.

(51) Int. Cl. A61K 31/44; C07C 69/76; A61K 31/216; C07C 69/78; A61P 35/00; A61P 29/00; A61P 9/00; C07D 271/08; A61K 31/4245; C07D 213/79

(52) U.S. Cl. 514/354; 560/85; 514/533; 560/81; 560/12; 548/125; 514/364; 546/326

(57) ABSTRACT

The present invention refers to new NO-donors aspirin derivatives, a process for their preparation and pharmaceutical compositions containing them.

The present invention refers to new nitric oxide (NO)-donor aspirin derivatives, a process for their preparation and pharmaceutical compositions containing them.

Aspirin is a well established drug belonging to the class of non steroidal anti-inflammatory drugs (NSAIDs) which displays a variety of actions including anti-inflammatory, analgesic, antipyretic and antithrombotic activities. The major drawback which limits its use is a relevant gastrotoxicity that is responsible for gastric ulceration, exacerbation of peptic ulcer symptoms, gastrointestinal hemorrhage and erosive gastritis (Goodman & Gilman's The Pharmacological Basis of Therapeutics. 10^{th} ed.; McGraw-Hill, Chapter 27).

WO 92/01668 discloses mononitrate aspirin derivatives having vasorelaxant and antianginal effects wherein the nitrooxy group is linked to the carboxylic group through a simple ester or amidic bond.

U.S. Pat. No. 5,859,053 discloses dinitrates of aspirin and their use for the alleviation of pain, inhibition of platelet aggregation, lowering of fever and for prevention of cardiovascular disorders.

WO 95/30641 and WO 97/16405 disclose new derivatives of aspirin wherein a moiety bearing a nitrooxy group is linked to the carboxylic group through a simple ester bond. These compounds have anti-inflammatory, analgesic and anti-thrombotic activity with lower gastrointestinal toxicity in comparison with aspirin.

J. Med. Chem. 2003, 46, 747-754 discloses a new series of NSAIDs in which aspirin is joined by an ester linkage to furoxan moieties. The products described present an anti-inflammatory trend, they are devoid of acute gastrotoxicity and show an antiplatelet activity. They do not behave as aspirin prodrugs in human serum.

J. F. Gilmer et al. Bioorg. Med. Chem. Lett., 17 (2007) 3217-3220 discloses the difficulty in designing aspirin ester prodrugs since aspirin already has an ester, acetate, which becomes highly labile in plasma when the carboxylic acid is converted to an ester. There is no evidence that the NO-aspirins known in the prior art are capable of significant aspirin release in human tissue.

The limit of these products is that they are rapidly metabolised in plasma and serum to salicylates without any formation of aspirin.

It was now object of the present invention to provide new (NO)-donor aspirin derivatives able to eliminate or at least reduce the drawbacks of the compounds known in the prior art.

On the basis of the known availability of certain acyloxyalkyl esters to undergo subsequently enzymatic and chemical metabolic cleavage (Bundgaard, H. “Design of prodrugs: bioreversible derivatives for various functional groups and chemical entities” in Design of prodrugs, Bundgaard, H. Ed. Elsevier, Amsterdam, 1985; Nielsen, N. M.; Bundgaard, H. “Evaluation of glycolamide esters and various other esters of aspirin as true aspirin prodrugs” J. Med. Chem. 1989, 32, 727-734), the applicant has developed a specific class of aspirin derivatives by conjugating acyloxyalkyl substructures containing either nitrooxy or furoxan NO-donor moieties to the carboxylic group of aspirin. These products have a very good aspirin release in human serum.

The compounds of the invention can be used for preventing and treating thrombotic cardiovascular events caused by platelet aggregation, thrombosis, and subsequent ischemic clinical events, including thrombotic or thromboembolic stroke, myocardial ischemia, myocardial infarction, angina pectoris, transient ischemic attack, reversible ischemic neurologic deficits, and any similar thrombotic event in any vascular bed (splanchnic, renal, aortic, peripheral, etc.).

The compounds of the invention are useful for the relief of pain, fever and inflammation of a variety of conditions including rheumatic fever, symptoms associated with influenza or other viral infections, common cold, low back and neck pain, dysmenorrhea, headache, toothache, sprains and strains, myositis, neuralgia, synovitis, arthritis, including rheumatoid arthritis degenerative joint diseases (osteoarthritis), gout and ankylosing spondylitis, bursitis, burns, injuries, following surgical and dental procedures.

The compounds of the invention can be used alone or in combination with NSAIDs, such as those described in Goodman and Gilman's, The Pharmacological Basis of Therapeutics, Tenth Edition, p. 687-716.

The compounds of the present invention are useful in the prevention and treatment of cancer diseases in particular those affecting gastrointestinal and urogenital apparatus, such as colon cancer, bladder cancer and prostate cancer.

Object of the present invention are compounds of general formula (I) and pharmaceutically acceptable salts or stereoisomers thereof:

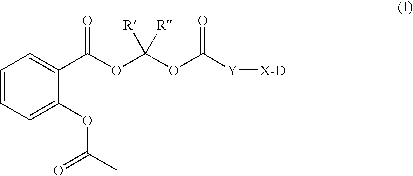

wherein:
R′ and R″ are independently H, straight or branched C₁-C₆ alkyl or when taken together R′ and R″ form a cycloalkyl from 3 to 7 carbon atoms;
Y is a bivalent radical having the following meanings:
a) straight or branched C₁-C₁₀ alkylene, optionally substituted with one or more of the substituents selected from the group consisting of: halogen atoms, hydroxy, —ONO₂, —OC(O) (C₁-C₁₀ alkyl)-ONO₂ and —O(C₁-C₁₀ alkyl)-ONO₂;
b)

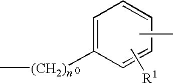

wherein R¹ is H, —COOH, —OH, CH₃ or Halogen, n⁰ is an integer from 0 to 10;
wherein the X moiety is not linked to —(CH₂)ₙ⁰;
c)

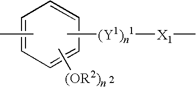

wherein:
n¹ is an integer from 0 to 1;
n² is an integer from 0 to 2;
Y¹ is —CH₂—CH₂— or —CH═CH— (CH₂)ₙ²—;

### X₁=—OCO— or —COO— and R² is H or CH₃;

wherein the X moiety is linked to X₁;
d)

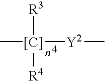

wherein:
n⁴ is an integer from 0 to 10;
R³ and R⁴ are the same or different, and are H or straight or branched C₁-C₆ alkyl;
wherein the X moiety is linked to Y²;
Y² is an heterocyclic saturated, unsaturated or aromatic 5 or 6 members ring, containing one or more heteroatoms selected from nitrogen, oxygen, sulfur,
and is selected from the group consisting of:

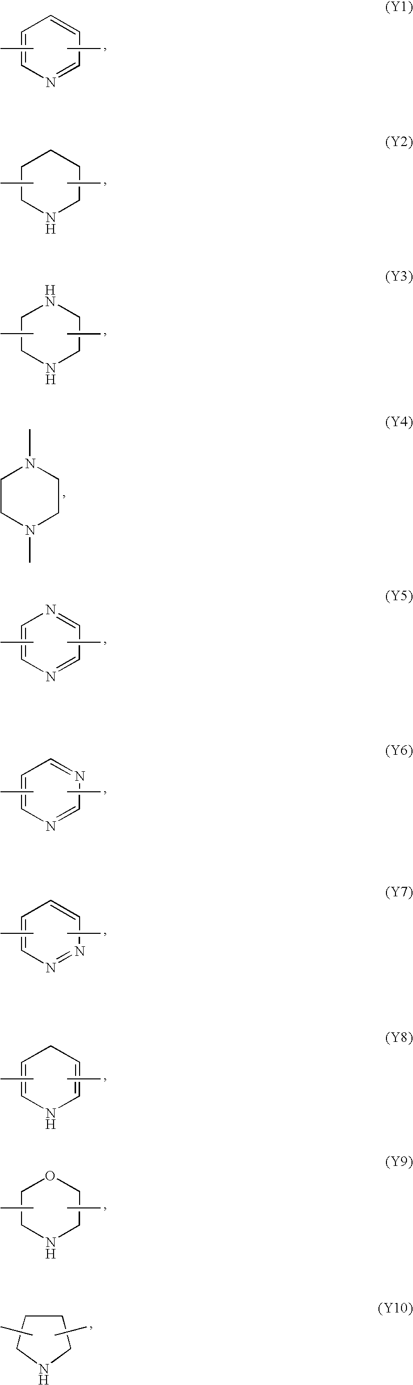

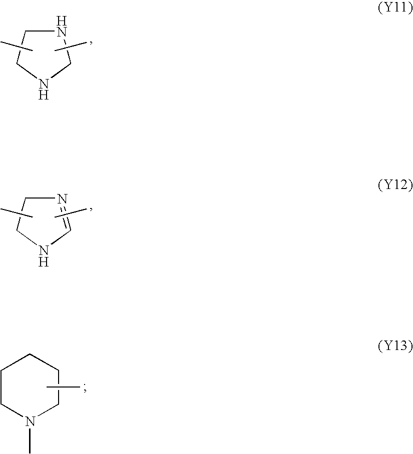

X is a moiety selected from the group consisting of: C₁-C₁₀ alkylene, —O—C₁-C₁₀ alkylene, —S—C₁-C₁₀ alkylene, —S(O)—C₁-C₁₀ alkylene and —S(O)₂—C₁-C₁₀ alkylene, optionally containing solubilising groups such as —OH, —NH₂, —COOH;
D=—ONO₂, C₁-C₁₀ alkyl substituted with one or more —ONO₂ groups, preferably —CH(ONO₂)CH₂ONO₂ or

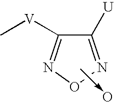

wherein V is —CH₂—, —O—, —S— or —NH—; U is C₁-C₁₀ alkyl, optionally substituted with —OH or —NH₂, aryl, C₁-C₁₀ alkoxy, aryloxy, C₁-C₁₀ thioalkyl, thioaryl, halogen, di-C₁-C₁₀ (alkylamino), diarylamino, aryl C₁-C₁₀ (alkylamino), C₁-C₁₀ (alkylsulphoxy), arylsulphoxy, C₁-C₁₀ (alkylsulphone), arylsulphone, —CN, —NO₂, —NHCOR₀, —COR₀, —COOR₀, —CON(R_{O})(R₁), wherein R₀ and R₁ are the same or different, and are H, C₁-C₁₀ alkyl or aryl.

The term “cycloalkyl” as used herein refers to a saturated or unsaturated cyclic hydrocarbon comprising from 3 to 7 carbon atoms such as cyclopropyl, cyclobutyl, cyclopentyl, cycloexyl and the like.

The term “C₁-C₁₀ alkyl” as used herein refers to branched or straight alkyl groups comprising 1 to 10 carbon atoms, including methyl, ethyl, n-propyl, isopropyl, n-butyl, isobutyl, t-butyl, pentyl, hexyl, octyl and the like.

The term “C₁-C₆” alkyl as used herein refers to branched or straight alkyl groups comprising 1 to 6 carbon atoms, including methyl, ethyl, n-propyl, isopropyl, n-butyl, isobutyl, t-butyl, pentyl, hexyl and the like.

The term “C₁-C₁₀ alkylene” as used herein refers to branched or straight C₁-C₁₀ hydrocarbon chain such as methylene, ethylene, propylene, isopropylene, n-butylene, pentylene, n-hexylene and the like.

The term “heterocyclic” as used herein refers to saturated, unsaturated or aromatic 5 or 6 members ring, containing one or more heteroatoms selected from nitrogen, oxygen, sulphur, such as for example pyridine, pyrazine, pyrimidine, pyrrolidine, morpholine, imidazole and the like.

The term “aryl group” refers to a mono or bicyclic carbocyclic ring system having one or two aromatic rings including phenyl, naphtyl and like. Aryl groups can be unsubstituted or substituted with one, two or three substituents independently selected from alkyl, alkoxy, amino, alkylamino, dialkylamino, arylamino, alkylarylamino hydroxyl, carboxyl, halogen atom and nitro.

The term “C₁-C₁₀ alkoxy” as used herein refers to R₂O—, wherein R₂ is an alkyl group as defined herein such as methoxy, ethoxy, n-propoxy, isopropoxy, n-butoxy, isobutoxy, t-butoxy, pentyloxy, hexyloxy, octyloxy and the like.

The term “aryloxy” as used herein refers to R₃O—, wherein R₃ is an aryl group as defined herein such as napthyloxy, quinolyloxy, isoquinolizinyloxy and the like.

The term “halogen” as used herein refers to fluorine, chlorine, bromine, iodine.

The term “thio” as used herein refers to —S—.

The term “alkylamino” as used herein refers to R₂NH—, wherein R₂ is an alkyl group as defined herein such as methylamino, ethylamino, butylamino and the like.

The term “dialkylamino” as used herein refers to R₂R₄N—, wherein R₂ and R₄ are independently an alkyl group as defined herein such as dimethylamino, diethylamino and the like.

The term “diarylamino” as used herein refers to R₃R₅N—, wherein R₃ and R₅ are independently an aryl group as defined herein.

The term “sulphoxy” as used herein refers to —S(O)—.

The term “sulphone” as used herein refers to —S(O)₂—.

As stated above, the invention includes also the pharmaceutically acceptable salts of the compounds of formula (I) and stereoisomers thereof.

Examples of pharmaceutically acceptable salts are either those with inorganic bases, such as sodium, potassium, calcium and aluminium hydroxides, or with organic bases, such as lysine, arginine, triethylamine, dibenzylamine, piperidine and other acceptable organic amines or bases as those reported for example in Wermuth, C. G. and Stahl, P. H. Pharmaceutical Salts:Properties, Selection, and Use—A Handbook Verlag Helvetica Chimica Acta, 2002.

The compounds according to the present invention, when they contain in the molecule one salifiable nitrogen atom, can be transformed into the corresponding salts by reaction, in an organic solvent such as acetonitrile, tetrahydrofuran, with the corresponding organic or inorganic acids.

Examples of organic acids are: oxalic, tartaric, maleic, succinic, citric acids. Examples of inorganic acids are: nitric, hydrochloric, sulphuric, phosphoric acids. Salts with nitric acid are preferred.

The compounds of the invention which have one or more asymmetric carbon atoms can exist as optically pure enantiomers, pure diastereomers, enantiomers mixtures, diastereomers mixtures, enantiomer racemic mixtures, racemates or racemate mixtures. Within the scope of the invention are also all the possible isomers, stereoisomers and their mixtures of the compounds of formula (I), including mixtures enriched in a particular isomer.

Preferred compounds of formula (I) are those, wherein:

Y is a bivalent radical having the following meanings:
R′ and R″ are independently H or straight or branched C₁-C₆ alkyl;
a) straight or branched C₁-C₁₀ alkylene;
b)

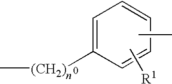

wherein R¹ is H, —COOH or —OH, n⁰ is an integer from 0 to 5;
wherein the X moiety is not linked to —(CH₂)n⁰;
d)

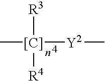

wherein:
n⁴ is an integer from 0 to 5;

### R³ and R⁴ are H;

wherein the X moiety is linked to Y²;
Y² is an heterocyclic selected from the group consisting of:

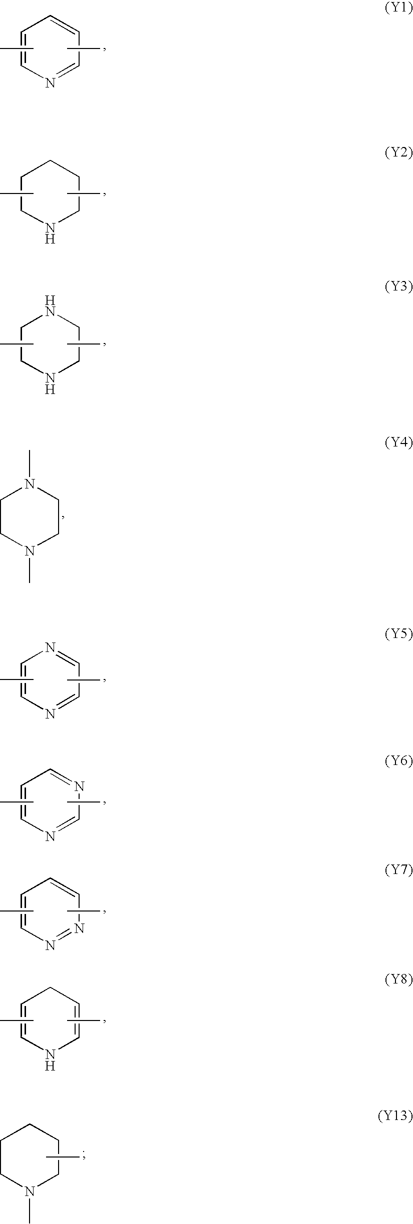

X is a moiety selected from the group consisting of: C₁-C₁₀ alkylene, —O—C₁-C₁₀ alkylene, —S—C₁-C₁₀ alkylene, —S(O)—C₁-C₁₀ alkylene and —S(O)₂—C₁-C₁₀ alkylene;
D has the same meanings reported above.

Particularly preferred compounds are compounds of formula (I) selected from the group consisting of:

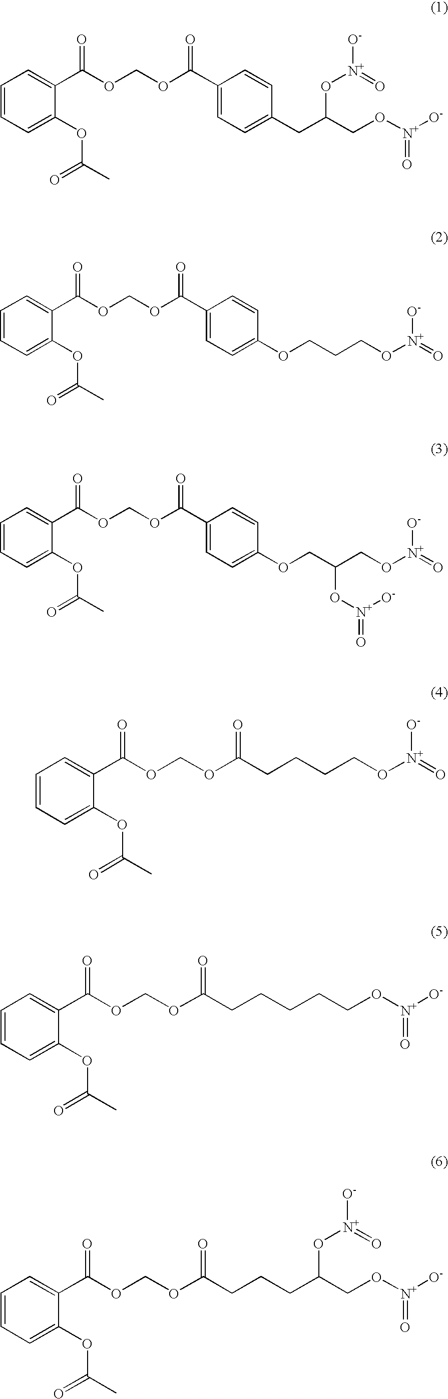

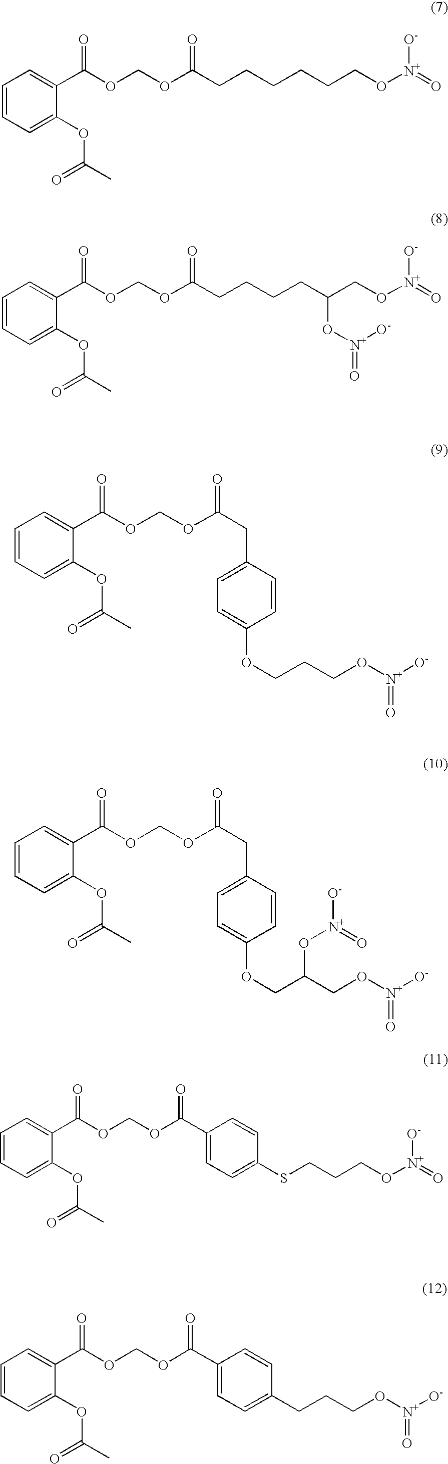

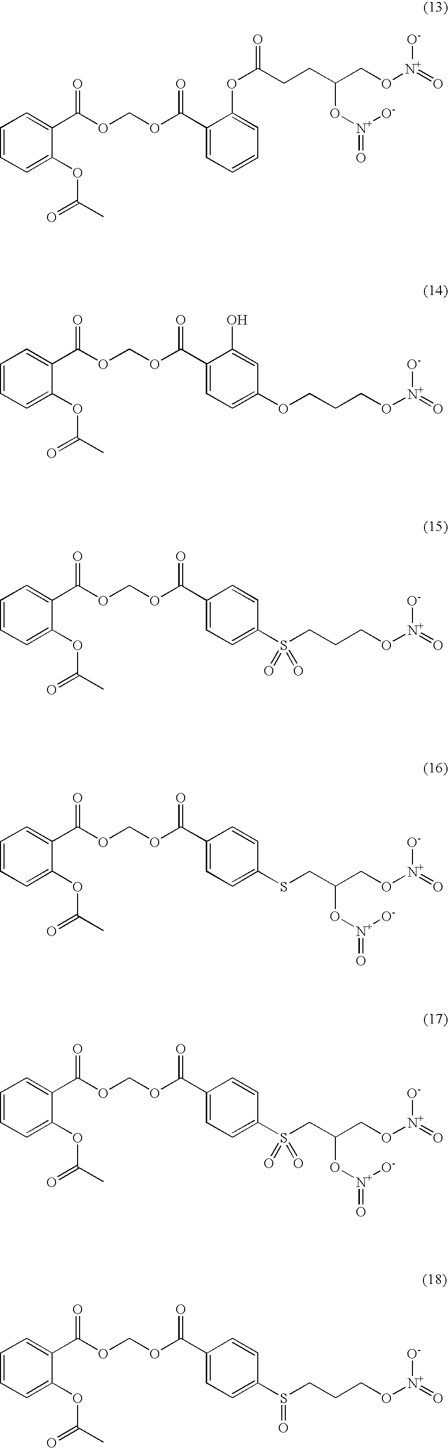

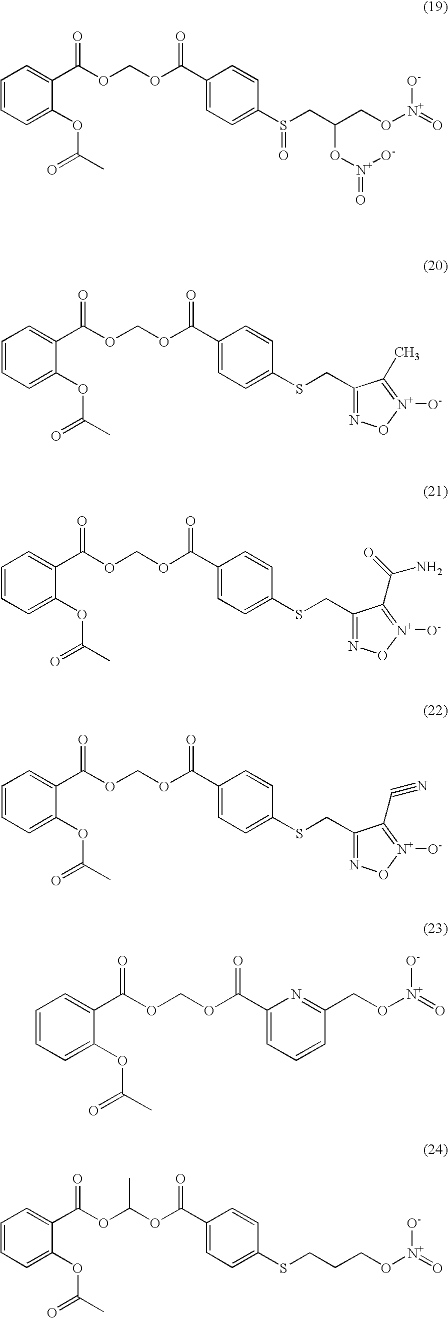

As mentioned above, object of the present invention are also pharmaceutical compositions containing at least a compound of the present invention of formula (I) together with non toxic adjuvants and/or carriers usually employed in the pharmaceutical field.

The daily dose of active ingredient that should be administered can be a single dose or it can be an effective amount divided into several smaller doses that are to be administered throughout the day. Usually, total daily dose may be in amounts preferably from 50 to 500 mg. The dosage regimen and administration frequency for treating the mentioned diseases with the compound of the invention and/or with the pharmaceutical compositions of the present invention will be selected in accordance with a variety of factors, including for example age, body weight, sex and medical condition of the patient as well as severity of the disease, route of administration, pharmacological considerations and eventual concomitant therapy with other drugs. In some instances, dosage levels below or above the aforesaid range and/or more frequent may be adequate, and this logically will be within the judgment of the physician and will depend on the disease state.

The compounds of the invention may be administered orally, parenterally, rectally or topically, by inhalation or aerosol, in formulations eventually containing conventional non-toxic pharmaceutically acceptable carriers, adjuvants and vehicles as desired. Topical administration may also involve the use of transdermal administration such as transdermal patches or iontophoresis devices. The term “parenteral” as used herein, includes subcutaneous injections, intravenous, intramuscular, intrasternal injection or infusion techniques.

Injectable preparations, for example sterile injectable aqueous or oleaginous suspensions may be formulated according to known art using suitable dispersing or wetting agents and suspending agents. The sterile injectable preparation may also be a sterile injectable solution or suspension in a non-toxic parenterally acceptable diluent or solvent. Among the acceptable vehicles and solvents are water, Ringer's solution and isotonic sodium chloride. In addition, sterile, fixed oils are conventionally employed as a solvent or suspending medium. For this purpose any bland fixed oil may be employed including synthetic mono or diglycerides, in addition fatty acids such as oleic acid find use in the preparation of injectables.

Suppositories for rectal administration of the drug can be prepared by mixing the active ingredient with a suitable non-irritating excipient, such as cocoa butter and polyethylene glycols.

Solid dosage forms for oral administration may include capsules, tablets, pills, powders, granules and gels. In such solid dosage forms, the active compound may be admixed with at least one inert diluent such as sucrose, lactose or starch. Such dosage forms may also comprise, as in normal practice, additional substances other than inert diluents, e.g. lubricating agents such as magnesium stearate. In the case of capsules, tablets and pills, the dosage forms may also comprise buffering agents. Tablets and pills can additionally be prepared with enteric coatings.

Liquid dosage forms for oral administration may include pharmaceutically acceptable emulsions, solutions, suspensions, syrups and elixirs containing inert diluents commonly used in the art, such as water. Such compositions may also comprise adjuvants, such as wetting agents, emulsifying and suspending agents, and sweetening, flavouring and the like.

### Experimental Procedures

### Synthetic Procedure

The compounds of formula (I) as above defined can be prepared by a process comprising the reaction of aspirin (1) with ClCH₂Br (2) in an organic solvent such as halogenated hydrocarbons, aromatic hydrocarbons, ethers, dipolar aprotic solvents, in particular tetrahydrofuran or N,N-dimethylformamide and mixtures thereof, in the presence of a suitable base such as a tertiary amine or K⁺t-BuO⁻ to give the chloromethyl ester (3) at a temperature between −40° C. and reflux temperature for a time between a few minutes and 72 hrs.

The intermediates (3) was treated with carboxylic acids (4) in a suitable organic solvent such as halogenated hydrocarbons, ethers, aromatic hydrocarbons, dipolar aprotic solvents, preferably DMF, in the presence of a suitable base such as tertiary amines, in particular triethylamine, or cesium carbonate to obtain the products (5).

The synthetic procedure was reported in Scheme 1:

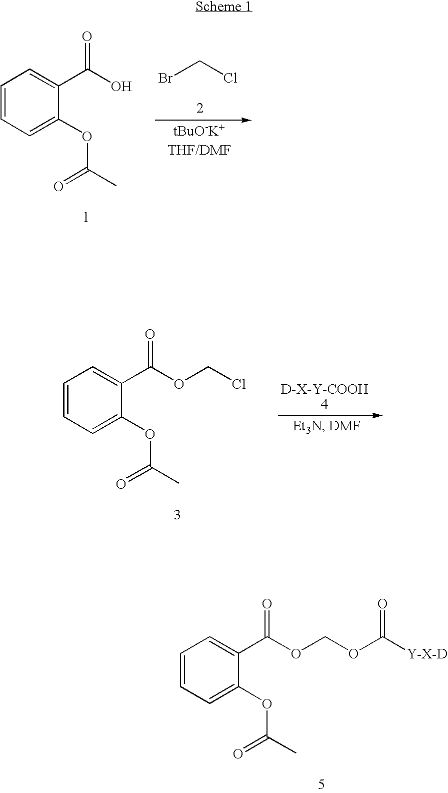

Compounds (4) wherein D is —ONO₂ can be obtained from the corresponding alcohols of formula HO—X—Y—COOH (II) by reaction with nitric acid and acetic anhydride in a temperature range from −50° C. to 0° C. or by reaction with N-Bromosuccinimide (NBS), triphenylphosphine (Ph₃P) and AgNO₃.

Moreover, compounds (4) wherein D is −ONO₂ can be obtained reacting a compound of formula L-X—Y—COOH (IIa) in which L is chlorine, bromine, iodide, tosylate, mesylate, trifluoromethanesulfonate and the like with silver nitrate in a suitable aprotic organic solvent such as acetone, tetrahydrofuran, acetonitrile, preferably acetonitrile.

The compounds (II) and (IIa) are commercially available or can be obtained by methods well known in the art.

Compounds (4) wherein D is the group (III):

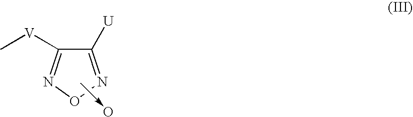

are prepared by the reaction of the appropriate bromomethylfuroxans in the presence of a suitable base such as a tertiary amine, in particular triethylamine, with p-substituted benzoic acid, in particular p-mercaptobenzoic acid.

Compounds (4) wherein D is —CH(ONO₂)CH₂ONO₂ can be prepared from the corresponding compounds of formula (IV) by treatment with iodine and silver nitrate in acetonitrile at a temperature between −20° C. and 80° C.:

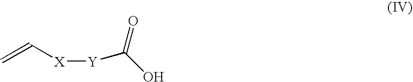

The compounds of formula (IV) are known compounds or can be obtained by methods well known in the art.

Finally, compounds (4) can be obtained from the corresponding aldehyde of formula D-X—Y—COH by reaction with a suitable oxidising agent such as potassium permanganate, sodium chlorite or sodium chlorite/H₂O₂ in a suitable organic solvent such acetic acid and the like at a temperature from 0 to 80° C. for a time from 1 minute to 72 hours.

Alternatively, the products (5) can be obtained by coupling directly the chloromethyl esters (6) to aspirin (1) in a suitable solvent such as DMF, in the presence of triethylamine.

The compounds (6) were obtained in conditions similar to those used to prepare (3) from (1) as above reported. The alternative synthetic procedure was reported in Scheme 2:

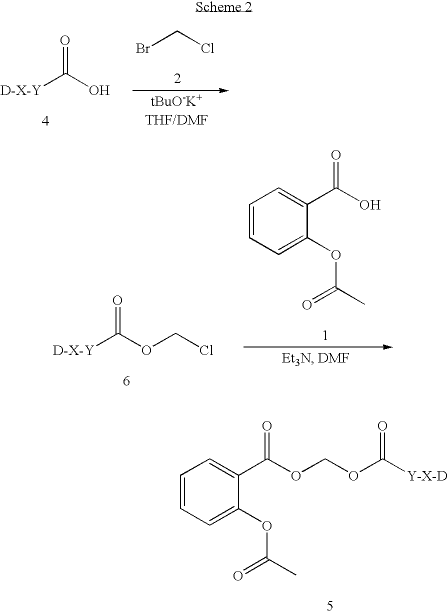

The 1-chloroethyl ester of aspirin (8) can be obtained by reacting the acylchloride of aspirin (7) with acetaldehyde in the presence of zinc chloride (WO 04/018484):

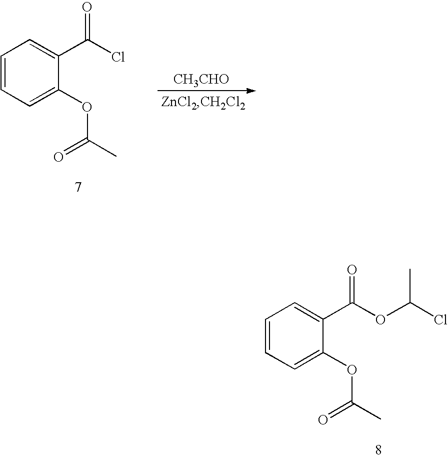

The products (5a) of the following formula:

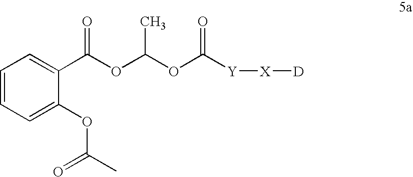

can be obtained coupling directly (8) with carboxylic acids (4) in a suitable organic solvent such as halogenated hydrocarbons, ethers, aromatic hydrocarbons, dipolar aprotic solvents, preferably DMF, in the presence of a suitable base such as tertiary amines, in particular triethyl amine, or cesium carbonate:

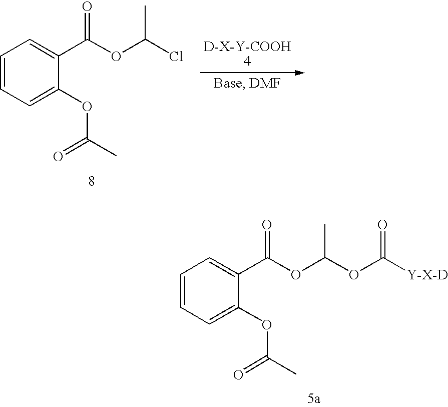

Alternatively, the products (5a) can be obtained by reacting compounds of formula D-X—Y—COO—CH(CH₃)Cl (7a) with aspirin (1) in the presence of cesium carbonate. Compounds (7a) can be obtained by reacting compounds of formula D-X—Y—COOH with zinc chloride in the presence of acetaldehyde (J. Med. Chem. 37(26), 4423, 1994).

### EXAMPLE 1

### ({4-[2,3-Bis(nitrooxy)propyl]benzoyl}oxy)methyl 2-(acetyloxy)benzoate: compound (1)

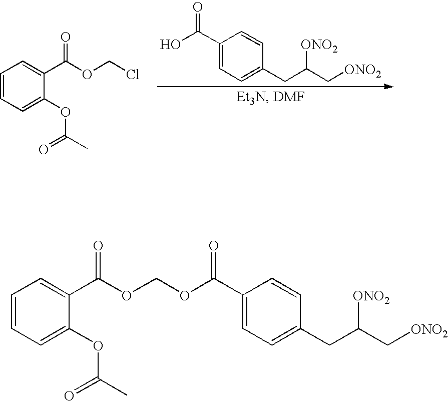

To a solution of chloromethyl 2-(acetyloxy)benzoate (1.20 g, 5.25 mmol; EP 136266) in dry DMF (30 mL) were added 4-[2,3-bis(nitrooxy)propyl]benzoic acid (1.50 g, 5.25 mmol; J. Pineal Res. 2007, 42, 371-385), Et₃N (0.75 mL, 5.25 mmol) and catalytic amount of KI. The mixture was stirred for 8 days, then was poured in H₂O (50 mL) and extracted with Et₂O (4×50 mL). The combined organic layers were washed with NaHCO₃ 1N (2×30 mL), dried with MgSO₄, filtered and concentrated under reduced pressure. The crude product was purified by flash chromatography (PE/EtOAc 9:1 to 8:2 v/v) to give the title compound (1.30 g) as colourless oil.

Yield 51%.

TLC: Rf=0.20 PE/EtOAc 80/20 v/v.

¹H-NMR (CDCl₃) δ 2.35 (3H, s), 3.03-3.18 (2H, m), 4.39-4.46 (1H, dd, AMX like system), 4.69-4.75 (1H, dd, AMX like system), 5.42-5.50 (1H, m), 6.19 (2H, s), 7.12 (1H, d, Arom), 7.26-7.35 (3H, m, Arom), 7.60 (1H, t, Arom), 8.06-8.10 (3H, m, Arom). ¹³C-NMR (CDCl₃) δ 21.0, 35.6, 60.4, 70.9, 80.1, 124.5, 126.2, 128.1, 129.9, 130.9, 132.2, 135.6, 141.4, 151.9, 159.6, 161.7, 169.5, 171.2. MS (CI) m/z 479 (M+1)⁺.

### EXAMPLE 2

### ({4-[3-(Nitrooxy)propoxy]benzoyl}oxy)methyl 2-(acetyloxy)benzoate: compound (2)

### Synthetic Procedure A

### 3-(4-Formylphenoxy)propyl nitrate

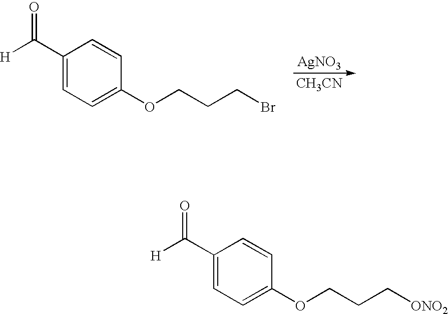

A solution of 4-(3-bromopropoxy)benzaldehyde (2.20 g, 9.05 mmol; Bioorg. Med. Chem. 2006, 14, 866-874) and AgNO₃ (3.10 g, 18.10 mmol) in CH₃CN (25 mL) was stirred at 70° C. for 1 h. Then brine was added to precipitate the excess of AgNO₃, the mixture was filtered through Celite and concentrated under reduced pressure. The residue was treated with CH₂Cl₂ (50 mL) and H₂O (50 mL). After separation the aqueous layer was extracted twice with CH₂Cl₂ (50 mL). The combined organic layers were dried with MgSO₄, filtered and concentrated under reduced pressure. The crude product was purified by flash chromatography (PE/EtOAc 90/10 v/v) to give the title compound as pale yellow oil (1.89 g).

Yield 93%.

TLC: Rf=0.14 PE/EtOAc 90/10 v/v.

¹H-NMR (CDCl₃) δ 2.26 (2H, qi), 4.16 (2H, t, J=6.0 Hz), 4.69 (2H, t, J=6.3 Hz), 7.00 (2H, d, Arom), 7.84 (2H, d, Arom), 9.89 (1H, s). ¹³C-NMR (CDCl₃) δ 26.8, 64.0, 69.7, 115.2, 130.3, 132.0, 163.4, 190.8. MS (CI) m/z 226 (M+1)⁺.

### 4-[3-(Nitrooxy)propoxy]benzoic acid

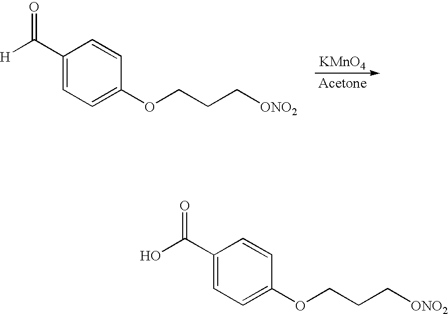

KMnO₄ (2.00 g, 12.58 mmol) was added. to a solution of 3-(4-formylphenoxy)propyl nitrate (1.89 g, 8.39 mmol) in acetone (25 mL), stirred at 0° C. The reaction was allowed to reach r.t. and it was completed after 1 h (TLC detection, eluent PE/EtOAc 70/30 v/v). Oxalic acid was added and the mixture was filtered and the filtrate was diluted with CH₂Cl₂ (50 mL). The organic layer was washed with H₂O (50 mL), then was dried with MgSO₄, filtered and concentrated under reduced pressure to give the title compound as white solid (1.64 g).

Yield 81%.

TLC: Rf=0.30 PE/EtOAc/HCOOH 80/20/0.1 v/v/v.

¹H-NMR (CDCl₃) δ 2.26 (2H, qi), 4.15 (2H, t), 4.69 (2H, t), 6.94 (2H, d, Arom), 8.08 (2H, d, Arom). MS (CI) m/z 242 (M+1)⁺.

### ({4-[3-(Nitrooxy)propoxy]benzoyl}oxy)methyl 2-(acetyloxy)benzoate

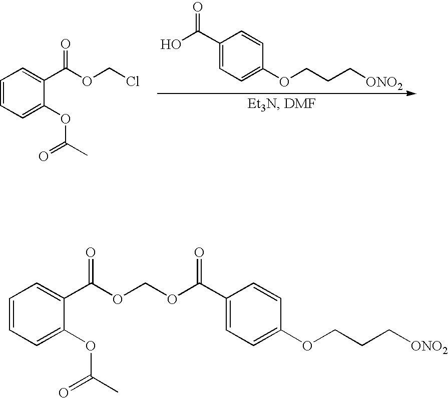

To a solution of chloromethyl 2-(acetyloxy)benzoate (0.95 g, 4.14 mmol) in dry DMF (20 mL) were added 4-[3-(nitrooxy)propoxy]benzoic acid (1.00 g, 4.14 mmol), Et₃N (0.58 mL, 4.14 mmol) and catalytic amount of KI. The mixture was stirred for 8 days, then was poured in H₂O (50 mL) and extracted with Et₂O (2×50 mL). The combined organic layers were washed with NaHCO₃ 1N (2×30 mL), dried with MgSO₄, filtered and concentrated under reduced pressure. The crude product was purified by flash chromatography (PE/EtOAc 8/2 v/v) to give the title compound (0.81 g) as colourless oil.

Yield 45%.

TLC: Rf=0.24 PE/EtOAc 80/20 v/v.

¹H-NMR (CDCl₃) δ 2.24 (2H, qi), 2.35 (3H, s), 4.13 (2H, t, J=6.0 Hz), 4.67 (2H, t, J=6.3 Hz), 6.17 (2H, s), 6.92 (2H, d, Arom), 7.11 (1H, d, Arom), 7.34 (1H, t, Arom), 7.59 (1H, t, Arom), 8.03-8.10 (3H, m, Arom). ¹³C-NMR (CDCl₃) δ 21.0, 26.8, 63.8, 69.6, 79.7, 114.2, 121.6, 122.1, 124.0, 126.1, 132.3, 134.6, 151.1, 162.8, 163.2, 164.8, 169.7. MS (CI) m/z 434 (M+1)⁺.

### Synthetic Procedure B

### Chloromethyl 4-[3-(nitrooxy)propoxy]benzoate

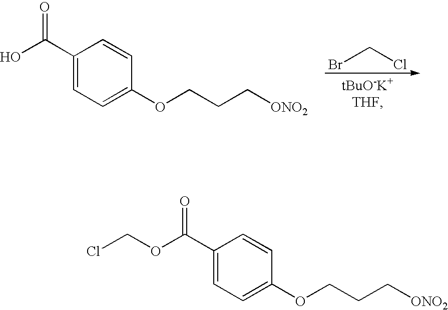

A solution of tBuO⁻K⁺ (0.23 g, 2 mmol) in dry THF (10 mL) was slowly added to a solution of 4-[3-(nitrooxy)propoxy]benzoic acid (0.48 g, 2 mmol) in dry THF (10 mL) stirred under N₂. One hour later dry DMF (10 mL) and bromochloromethane (13 mL, 100 eq) were added and the resulting mixture was stirred for 48 hours. Then the mixture was poured in a saturated solution of NH₄Cl (50 mL) and the layers separated. The organic layer was dried with MgSO₄, filtered and concentrated under reduced pressure. The crude product was purified by flash chromatography (PE/EtOAc 8/2 v/v) to give the title compound (0.30 g) as colourless oil.

Yield 39%.

TLC: Rf=0.55 PE/EtOAc 80/20 v/v.

¹H-NMR (CDCl₃) δ 2.25 (2H, qi), 4.14 (2H, t, J=5.7 Hz), 4.68 (2H, t, J=6.3 Hz), 5.94 (2H, s), 6.93 (2H, d, Arom), 8.03 (2H, d, Arom). MS (CI) m/z 290 (M+1)⁺.

### ({4-[3-(Nitrooxy)propoxy]benzoyl}oxy)methyl 2-(acetyloxy)benzoate

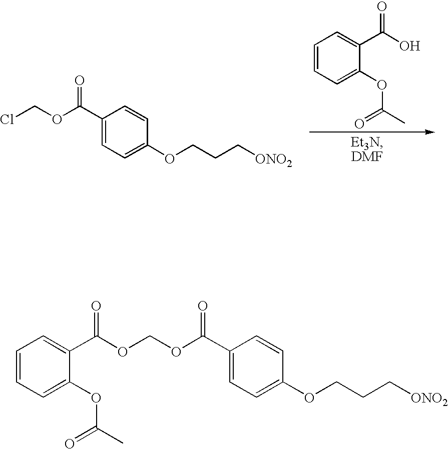

To a solution of chloromethyl 4-[3-(nitrooxy)propoxy]benzoate (0.21 g, 0.74 mmol) in dry DMF (5 mL) were added 2-acetyloxybenzoic acid (0.13 g, 0.74 mmol), Et₃N (0.10 mL, 0.74 mmol) and catalytic amount of KI. The mixture was stirred for 7 days, then was poured in H₂O (15 mL) and extracted with Et₂O (4×15 mL). The combined organic layers were washed with NaHCO₃ 1N (2×15 mL), dried with MgSO₄, filtered and concentrated under reduced pressure. The crude product was purified by flash chromatography (PE/EtOAc 8/2 v/v) to give the title compound (0.17 g) as colourless oil.

Yield 53%.

TLC: Rf=0.24 PE/EtOAc 80/20 v/v.

¹H-NMR (CDCl₃) δ 2.24 (2H, qi), 2.35 (3H, s), 4.13 (2H, t, J=6.0 Hz), 4.67 (2H, t, J=6.3 Hz), 6.17 (2H, s), 6.92 (2H, d, Arom), 7.11 (1H, d, Arom), 7.34 (1H, t, Arom), 7.59 (1H, t, Arom), 8.03-8.10 (3H, m, Arom). ¹³C-NMR (CDCl₃) δ 21.0, 26.8, 63.8, 69.6, 79.7, 114.2, 121.6, 122.1, 124.0, 126.1, 132.3, 134.6, 151.1, 162.8, 163.2, 164.8, 169.7. MS (CI) m/z 434 (M+1)⁺.

### EXAMPLE 3

### ({4-[2,3-Bis(nitrooxy)propoxy]benzoyl}oxy)methyl 2-(acetyloxy)benzoate: compound (3)

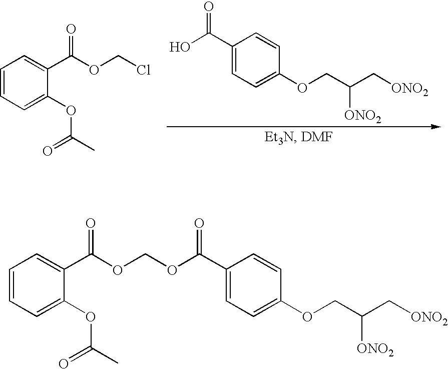

To a solution of chloromethyl 2-(acetyloxy)benzoate (0.60 g, 2.62 mmol) in dry DMF (10 mL) were added 4-[2,3-bis(nitrooxy)propoxy]benzoic acid (0.79 g, 2.62 mmol; J. Pineal Res. 2007, 42, 371-385), Et₃N (0.36 mL, 2.62 mmol) and catalytic amount of KI. The mixture was stirred for 8 days, then was poured in H₂O (25 mL) and extracted with Et₂O (3×25 mL). The combined organic layers were washed with NaHCO₃ 1N (3×20 mL), dried with MgSO₄, filtered and concentrated under reduced pressure. The crude product was purified by flash chromatography (PE/EtOAc 8/2 v/v) to give the title compound (0.52 g) as colourless oil.

Yield 33%.

TLC: Rf=0.45 PE/EtOAc 80/20 v/v.

¹H-NMR (CDCl₃) δ 2.35 (3H, s), 4.31 (2H, d), 4.75-4.81 (1H, dd, AMX like system), 4.90-4.96 (1H, dd, AMX like system), 5.30-5.64 (1H, m), 6.17 (2H, s), 6.93 (2H, d, Arom), 7.13 (1H, d, Arom), 7.32 (1H, t, Arom), 7.59 (1H, t, Arom), 8.05-8.09 (3H, m, Arom). ¹³C-NMR (CDCl₃) δ 21.0, 64.7, 68.6, 76.4, 79.8, 114.2, 122.0, 122.7, 124.0, 126.1, 132.2, 132.2, 134.3, 151.0, 161.6, 163.1, 164.6, 169.7. MS (CI) m/z 495 (M+1)⁺.

### EXAMPLE 4

### {[(5-(Nitrooxy)pentanoyl]oxy}methyl 2-(acetyloxy)benzoate: compound (4)

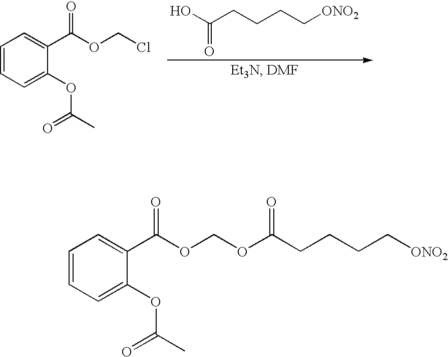

To a solution of chloromethyl 2-(acetyloxy)benzoate (1.00 g, 4.37 mmol) in dry DMF (10 mL) were added 5-nitrooxypentanoic acid (0.71 g, 4.37 mmol; J. Med. Chem. 2005, 48, 1322-1329), Et₃N (0.61 mL, 4.37 mmol) and catalytic amount of KI. The mixture was stirred for 10 days, then was poured in H₂O (25 ml) and extracted with Et₂O (3×25 mL). The combined organic layers were dried with MgSO₄, filtered and concentrated under reduced pressure. The crude product was purified by flash chromatography (PE/EtOAc 8/2 v/v) to give the title compound (0.15 g) as colourless oil.

Yield 10%.

TLC: Rf=0.35 PE/EtOAc 80/20 v/v.

¹H-NMR (CDCl₃) δ 1.72-1.80 (4H, m), 2.36 (3H, s), 2.43-2.46 (2H, m), 4.43-4.47 (2H, m), 5.95 (2H, s), 7.12 (1H, d, Arom), 7.34 (1H, t, Arom), 7.61 (1H, t, Arom), 8.07 (1H, d, Arom). ¹³C-NMR (CDCl₃) δ 20.5, 21.0, 26.1, 33.2, 72.6, 79.3, 121.9, 124.0, 126.2, 132.2, 134.7, 151.1, 163.0, 169.7, 171.6. MS (CI) m/z 356 (M+1)⁺.

### EXAMPLE 5

### {[6-(Nitrooxy)hexanoyl]oxy}methyl 2-(acetyloxy)benzoate: compound (5)

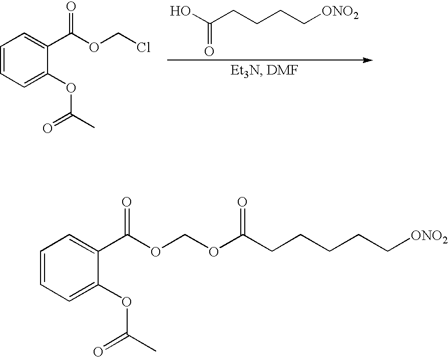

To a solution of chloromethyl 2-(acetyloxy)benzoate (1.00 g, 4.37 mmol) in dry DMF (10 mL) were added 6-nitrooxyhexanoic acid (0.77 g, 4.37 mmol; US2006189603), Et₃N (0.61 mL, 4.37 mmol) and catalytic amount of KI. The mixture was stirred for 10 days, then was poured in H₂O (25 mL) and extracted with Et₂O (3×25 mL). The combined organic layers were dried with MgSO₄, filtered and concentrated under reduced pressure. The crude product was purified by flash chromatography (PE/EtOAc 8/2 v/v) to give the title compound (0.29 g) as colourless oil.

Yield 17%.

TLC: Rf=0.38 PE/EtOAc 80/20 v/v.

¹H-NMR (CDCl₃) δ 1.41-1.49 (2H, m), 1.64-1.78 (4H, m), 2.36 (3H, s), 2.41 (2H, t, J=7.2 Hz), 4.41 (2H, t, J=6.6 Hz), 5.95 (2H, s), 7.11 (1H, d, Arom), 7.34 (1H, t, Arom), 7.61 (1H, t, Arom), 8.07 (1H, d, Arom). ¹³C-NMR (CDCl₃) δ 21.2, 24.3, 25.3, 26.7, 33.8, 73.1, 79.4, 122.2, 124.3, 126.4, 132.4, 135.0, 151.4, 163.2, 169.9, 172.2. MS (CI) m/z 370 (M+1)⁺.

### EXAMPLE 6

### {[5,6-Bis(nitrooxy)hexanoyl]oxy}methyl 2-(acetyloxy)benzoate: compound (6)

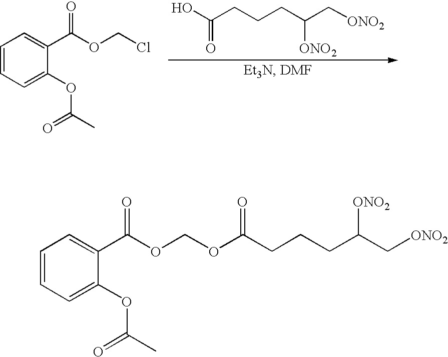

To a solution of chloromethyl 2-(acetyloxy)benzoate (0.30 g, 1.31 mmol) in dry DMF (3 mL) were added 5,6-bis(nitrooxy)hexanoic acid (0.31 g, 1.31 mmol; J. Med. Chem. 2005, 48, 1322-1329), Et₃N (0.19 mL, 1.31 mmol) and catalytic amount of KI. The mixture was stirred for 10 days, then was poured in H₂O (30 mL) and extracted with Et₂O (3×25 mL). The combined organic layers were washed with NaHCO₃ 1N (2×30 mL), dried with MgSO₄, filtered and concentrated under reduced pressure. The crude product was purified by flash chromatography (PE/EtOAc 9/1 to 8/2 v/v) to give the title compound (0.19 g) as colourless oil.

Yield 33%.

TLC: Rf=0.26 PE/EtOAc 80/20 v/v.

¹H-NMR (CDCl₃) δ 1.74-1.83 (4H, m), 2.36 (3H, s), 2.45-2.48 (2H, m), 4.39-4.46 (1H, dd, AMX like system), 4.68-4.74 (1H, dd, AMX like system), 5.25-5.28 (1H, m), 5.95 (2H, s), 7.10 (1H, d, Arom), 7.32 (1H, t, Arom), 7.62 (1H, t, Arom), 8.07 (1H, d, Arom). ¹³C-NMR (CDCl₃) δ 20.0, 21.0, 28.4, 33.0, 71.0, 78.7, 79.2, 121.8, 124.0, 126.2, 132.2, 134.9, 151.1, 163.0, 169.6, 171.3. MS (CI) m/z 431 (M+1)⁺.

### EXAMPLE 7

### {[7-(Nitrooxy)heptanoyl]oxy}methyl 2-(acetyloxy)benzoate: compound (7)

### 6-Nitrooxyheptanoic acid

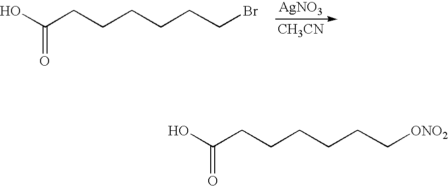

A solution of 7-bromohexanoic acid (1.80 g, 8.61 mmol; J. Am. Chem. Soc. 1947, 69, 2466) in CH₃CN (15 mL) was added to a solution of AgNO₃ (4.40 g, 25.83 mmol) in CH₃CN (15 mL) stirred at 50° C. At the end of the addition the mixture was heated at 70° C. for 12 hours. Then brine was added to precipitate the excess of AgNO₃, the mixture was filtered through Celite and concentrated under reduced pressure. The residue was treated with CH₂Cl₂ (20 mL) and H₂O (20 mL). After separation the aqueous layer was extracted twice with CH₂Cl₂ (20 mL). The combined organic layers were dried with MgSO₄, filtered and concentrated under reduced pressure to give the title compound as pale yellow oil (1.34 g).

Yield 81%.

TLC: Rf=0.61 PE/EtOAc 60/40 v/v.

¹H-NMR (CDCl₃) δ 1.43-1.49 (4H, m), 1.61-1.78 (4H, m), 2.37 (2H, t, J=7.2 Hz), 4.45 (2H, t, J=6.6 Hz). ¹³C-NMR (CDCl₃) δ 24.3, 25.3, 26.5, 28.5, 33.8, 73.2, 180.1. MS (CI) m/z 192 (M+1)⁺.

### {[7-(Nitrooxy)heptanoyl]oxy}methyl 2-(acetyloxy)benzoate

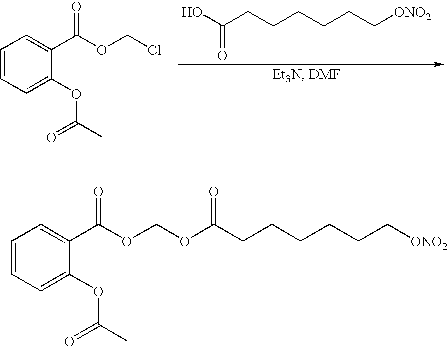

To a solution of chloromethyl 2-(acetyloxy)benzoate (1.00 g, 4.37 mmol) in dry DMF (10 mL) were added 7-nitrooxyhexanoic acid (0.83 g, 4.37 mmol), Et₃N (0.61 mL, 4.37 mmol) and catalytic amount of KI. The mixture was stirred for 10 days, then was poured in H₂O (25 mL) and extracted with Et₂O (3×25 mL). The combined organic layers were dried with MgSO₄, filtered and concentrated under reduced pressure. The crude product was purified by flash chromatography (PE/EtOAc 8/2 v/v) to give the title compound (0.38 g) as colourless oil.

Yield 17%.

TLC: Rf=0.36 PE/EtOAc 80/20 v/v.

¹H-NMR (CDCl₃) δ 1.34-1.43 (4H, m), 1.61-1.71 (4H, m), 2.36-2.42 (5H, m), 4.41 (2H, t, J=6.6 Hz), 5.95 (2H, s), 7.11 (1H, d, Arom), 7.34 (1H, t, Arom), 7.60 (1H, t, Arom), 8.08 (1H, d, Arom). ¹³C-NMR (CDCl₃) δ 21.0, 24.3, 25.3, 26.5, 28.4, 33.7, 73.2, 79.2, 122.0, 124.1, 126.2, 132.2, 134.8, 151.2, 163.0, 169.7, 171.1. MS (CI) m/z 384 (M+1)⁺.

### EXAMPLE 8

### {[6,7-Bis(nitrooxy)heptanoyl]oxy}methyl 2-(acetyloxy)benzoate: compound (8)

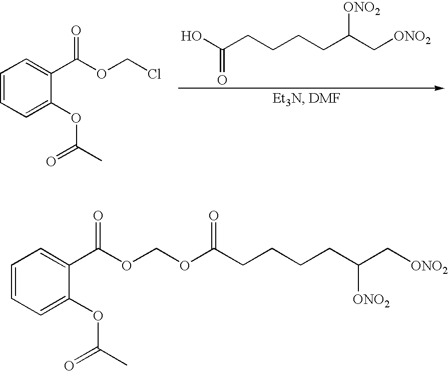

To a solution of chloromethyl 2-(acetyloxy)benzoate (0.20 g, 0.87 mmol) in dry DMF (3 mL) were added 6,7-bis(nitrooxy)hexanoic acid (0.33 g, 0.87 mmol), Et₃N (0.13 mL, 0.87 mmol) and catalytic amount of KI. The mixture was stirred for 10 days, then was poured in H₂O (30 mL) and extracted with Et₂O (3×25 mL). The combined organic layers were washed with NaHCO₃ 1N (2×30 mL), dried with MgSO₄, filtered and concentrated under reduced pressure. The crude product was purified by flash chromatography (PE/EtOAc 9/1 to 8/2 v/v) to give the title compound (0.12 g) as colourless oil.

Yield 27%.

TLC: Rf=0.31 PE/EtOAc 80/20 v/v.

¹H-NMR (CDCl₃) δ 1.45-1.51 (2H, m), 1.67-1.76 (4H, m), 2.35 (3H, s), 2.42 (2H, t, J=6.9 Hz), 4.38-4.45 (1H, dd, AMX like system), 4.67-4.72 (1H, dd, AMX like system), 5.15-5.28 (1H, m), 5.95 (2H, s), 7.11 (1H, d, Arom), 7.34 (1H, t, Arom), 7.60 (1H, t, Arom), 8.08 (1H, d, Arom). ¹³C-NMR (CDCl₃) δ 21.0, 24.0, 24.2, 29.0, 33.4, 71.1, 78.9, 79.2, 121.9, 124.0, 126.1, 132.2, 134.8, 151.1, 163.0, 169.7, 171.8. MS (CI) m/z 445 (M+1)⁺.

### EXAMPLE 9

### ({4-[3-(Nitrooxy)propoxy]benzoyl}oxy)methyl 2-(acetyloxy)benzoate: compound (9)

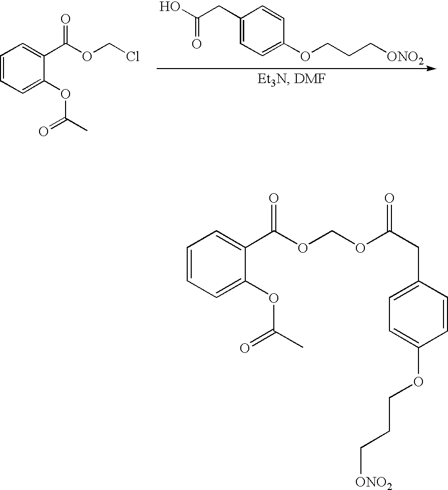

To a solution of chloromethyl 2-(acetyloxy)benzoate (0.60 g, 2.62 mmol) in dry DMF (10 mL) were added {4-[3-(nitrooxy)propoxy]phenyl}acetic acid (0.67 g, 2.62 mmol), Et₃N (0.37 mL, 2.62 mmol) and catalytic amount of KI. The mixture was stirred for 8 days, then was poured in H₂O (30 mL) and extracted with Et₂O (4×30 mL). The combined organic layers were washed with NaHCO₃ 1N (2×25 mL), dried with MgSO₄, filtered and concentrated under reduced pressure. The crude product was purified by flash chromatography (PE/EtOAc 9/1 v/v) to give the title compound (0.22 g) as colourless oil.

Yield 21%.

TLC: Rf=0.30 PE/EtOAc 80/20 v/v.

¹H-NMR (CDCl₃) δ 2.19 (2H, qi), 2.33 (3H, s), 3.54 (2H, s), 4.03 (2H, t, J=5.7 Hz), 4.66 (2H, t, J=6.3 Hz), 5.95 (2H, s), 6.83 (2H, d, Arom), 7.12 (1H, d, Arom), 7.19 (2H, d, Arom), 7.31 (1H, t, Arom), 7.60 (1H, t, Arom), 8.01 (1H, d, Arom). ¹³C-NMR (CDCl₃) δ 20.9, 27.0, 40.0, 63.5, 70.0, 79.5, 114.6, 122.0, 124.0, 125.6, 126.1, 130.4, 132.2, 134.7, 151.1, 157.7, 163.0, 169.7, 170.6. MS (CI) m/z 448 (M+1)⁺.

### EXAMPLE 10

### [(2-{4-[2,3-Bis(nitrooxy)propoxy]phenyl}acetyl)oxy]methyl 2-(acetyloxy)benzoate: compound (10)

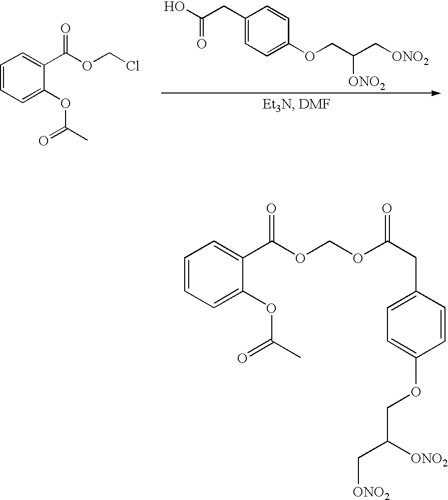

To a solution of chloromethyl 2-(acetyloxy)benzoate (0.60 g, 2.62 mmol) in dry DMF (10 mL) were added {4-[2,3-bis(nitrooxy)propoxy]phenyl}acetic acid (0.83 g, 2.62 mmol), Et₃N (0.37 mL, 2.62 mmol) and catalytic amount of KI. The mixture was stirred for 10 days, then was poured in H₂O (30 mL) and extracted with Et₂O (4×30 mL). The combined organic layers were washed with NaHCO₃ 1N (2×30 mL), dried with MgSO₄, filtered and concentrated under reduced pressure. The crude product was purified by flash chromatography (PE/Acetone 9/1 v/v) to give the title compound (0.51 g) as colourless oil.

Yield 38%.

TLC: Rf=0.24 PE/EtOAc 80/20 v/v.

¹H-NMR (CDCl₃) δ 2.32 (3H, s), 3.64 (2H, s), 4.19 (2H, d), 4.73-4.79 (1H, dd, AMX like system), 4.88-4.93 (1H, dd, AMX like system), 5.56-5.59 (1H, m), 5.95 (2H, s), 6.84 (2H, d Arom), 7.12 (1H, d, Arom), 7.21 (2H, d, Arom), 7.31 (1H, t, Arom), 7.62 (1H, t, Arom), 8.00 (1H, d, Arom). ¹³C-NMR (CDCl₃) δ 21.0, 40.0, 64.7, 68.8, 76.7, 79.5, 114.7, 121.9, 124.0, 126.1, 126.7, 130.7, 132.2, 134.7, 151.1, 156.8, 162.9, 169.7, 170.4. MS (CI) m/z 509 (M+1)⁺.

### EXAMPLE 11

### [(4-{[3-(Nitrooxy)propyl]thio}benzoyl)oxy]methyl 2-(acetyloxy)benzoate: compound (11)

### 4-[(3-Bromopropyl)thio]benzoic acid

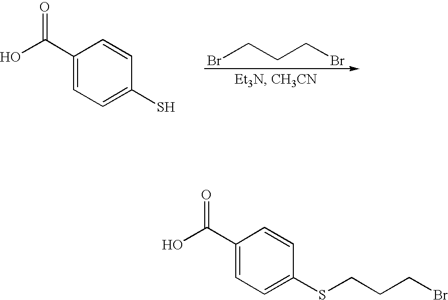

To a sospension of 4-mercaptobenzoic acid 90% (3.0 g, 17.50 mmol) in CH₃CN (30 mL), stirred at 0° C., 1,3-dibromopropane (9.0 mL, 87.50 mmol) and Et₃N (5.00 mL, 35.0 mmol) were added. After 3 hours the reaction was completed. The mixture was poured in HCl 1M (30 mL) and extracted with CH₂Cl₂ (3×40 mL); the combined organic layers were washed with brine (30 mL), dried with MgSO₄. filtered and concentrated under reduced pressure. The crude product was purified by flash chromatography (PE/EtOAc/HCOOH 90/10/0.1 to 70/30/0.1 v/v/v) to give the title compound (3.32 g) as white solid.

Yield 70%.

TLC: Rf=0.38 PE/EtOAc/HCOOH 80/20/0.1 v/v/v.

¹H-NMR (CD₃OD) δ 2.18 (2H, qi), 3.18 (2H, t, J=6.9 Hz), 3.57 (2H, t, J=7.2 Hz), 7.37 (2H, d, Arom), 7.92 (2H, d, Arom). ¹³C-NMR (CD₃OD) δ 30.9, 32.5, 32.9, 127.7, 128.7, 131.0, 144.7, 169.5. MS (CI) m/z 275/277 (M+1)⁺.

### 4-{[3-(Nitrooxy)propyl]thio}benzoic acid

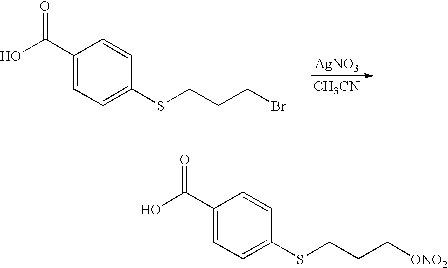

A solution of 4-[(3-bromopropyl)thio]benzoic acid (2.70 g, 10.0 mmol) and AgNO₃ (3.40 g, 20.0 mmol) in CH₃CN (50 mL) was stirred at 70° C. for 5 h. Then brine was added to precipitate the excess of AgNO₃, the mixture was filtered through Celite and concentrated under reduced pressure. The residue was treated with CH₂Cl₂ (50 mL) and H₂O (50 mL). After separation the aqueous layer was extracted twice with CH₂Cl₂ (50 mL). The combined organic layers were dried with MgSO₄, filtered and concentrated under reduced pressure. The crude product was purified by flash chromatography (PE/EtOAc/HCOOH 80/20/0.1 v/v/v) to give the title compound as white solid (1.90 g).

Yield 80%.

TLC: Rf=0.28 PE/EtOAc/HCOOH 80/20/0.1 v/v/v.

¹H-NMR (CD₃OD) δ 2.07 (2H, qi), 3.13 (2H, t, J=7.2 Hz), 4.60 (2H, t, J=6.3 Hz), 7.38 (2H, d, Arom), 7.93 (2H, d, Arom). ¹³C-NMR (CD₃OD) δ 27.4, 28.9, 72.8, 127.9, 128.8, 131.3, 144.4, 169.4. MS (CI) m/z 258 (M+1)⁺.

### [(4-{[3-(Nitrooxy)propyl]thio}benzoyl)oxy]methyl 2-(acetyloxy)benzoate

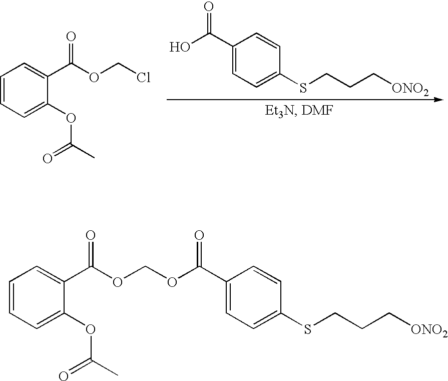

To a solution of chloromethyl 2-(acetyloxy)benzoate (0.35 g, 1.55 mmol) in dry DMF (10 mL) were added 4-{[3-(nitrooxy)propyl]thio}benzoic acid (0.40 g, 1.55 mmol), Et₃N (0.22 mL, 1.55 mmol) and catalytic amount of KI. The mixture was stirred for 10 days, then was poured in H₂O (30 mL) and extracted with Et₂O (3×30 mL). The combined organic layers were washed with NaHCO₃ 1N (2×30 mL), dried with MgSO₄, filtered and concentrated under reduced pressure. The crude product was purified by flash chromatography (PE/EtOAc 8/2 v/v) to give the title compound (0.19 g) as colourless oil.

Yield 40%.

TLC: Rf=0.32 PE/EtOAc 80/20 v/v.

¹H-NMR (CDCl₃) δ 2.11 (2H, qi), 2.35 (3H, s), 3.10 (2H, t, J=7.2 Hz), 4.58 (2H, t, J=6.3 Hz), 6.17 (2H, s), 7.12 (1H, d, Arom), 7.33-7.35 (3H, m, Arom), 7.60 (1H, t, Arom), 7.99 (2H, d, Arom), 8.09 (1H, d, Arom). ¹³C-NMR (CDCl₃) δ 21.0, 26.1, 28.1, 71.0, 79.8, 122.0, 124.0, 125.9, 126.1, 126.9, 130.6, 132.2, 134.7, 143.7, 151.1, 161.9, 163.1, 164.8, 169.3, 169.7. MS (CI) m/z 450 (M+1)⁺.

Alternatively, the compound [(4-{[3-(nitrooxy)propyl]thio}benzoyl)oxy]methyl 2-(acetyloxy)benzoate can be obtained with the following procedure:

To a solution of chloromethyl 2-(acetyloxy)benzoate (0.50 g, 2.19 mmol) in dry DMF (10 mL) were added 4-{[3-(nitrooxy)propyl]thio}benzoic acid (0.56 g, 2.19 mmol) and cesium carbonate (0.36 g, 1.1 mmol). The mixture was stirred for 24 hours then was poured in H₂O (30 mL) and extracted with Et₂O (3×20 mL). The combined organic layers were washed twice with a saturated solution of NaHCO₃ (20 mL), dried with MgSO₄, filtered and concentrated under reduced pressure. The crude product was purified by flash chromatography (PE/EtOAc 9/1 v/v) to give the title compound (0.61 g) as a colourless oil.

Yield 62%.

### EXAMPLE 12

### ({4-[3-(Nitrooxy)propyl]benzoyl}oxy)methyl 2-(acetyloxy)benzoate: compound (12)

### 3-[4-(1,3-Dioxolan-2-yl)phenyl]propan-1-ol

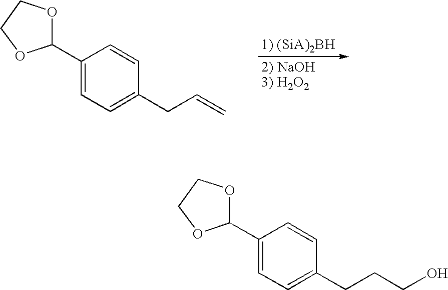

A solution of NaBH₄ (6.5 g, 0.17 mol) in dry THF (150 mL) was slowly added to amylene (66 mL, 0.62 mol) stirred at 0° C. Then BF₃.Et₂O (15 mL, 0.12 mol) was added in 30 min to the mixture maintained at 0° C. (J. Chem. Soc. Perkin Trans. I 1985, 1627-1635). After 5.5 hours a solution of 2-[4-(allyloxy)phenyl]-1,3-dioxolane (3.75 g, 12.3 mmol) in dry THF (40 mL) was slowly added and the stirring was continued for 24 hours. Then to the mixture, cooled at 0° C., H₂O (80 mL), NaOH 3M (80 mL) and H₂O₂ 30% (120 mL) were added and the resulting mixture was heated at 40° C. for 1.5 hours. After separation the organic layer was washed with H₂O (50 mL), dried with MgSO₄, filtered and concentrated under reduced pressure. The crude product so obtained was purified by flash chromatography (PE/EtOAc 8/2 to 6/4 v/v) to give the title compound as colourless oil (2.52 g).

Yield 98%.

### 3-[4-(1,3-Dioxolan-2-yl)phenyl]propyl nitrate

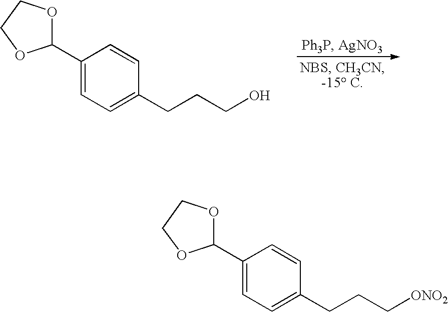

Ph₃P (0.79 g, 3 mmol), AgNO₃ (0.61 g, 3.6 mmol) and NBS (0.47 g, 2.64 mmol) were added to a solution of 3-[4-(1,3-dioxolan-2-yl)phenyl]propan-1-ol (0.50 g, 2.4 mmol) in dry CH₃CN (10 mL), stirred at −15° C. 2 hours later the mixture was allowed to reach room temperature, then filtered. The filtrate was extracted twice with CH₂Cl₂ (40 mL); the organic layers were dried with MgSO₄, filtered and concentrated under reduced pressure. The crude product so obtained was purified by flash chromatography (PE/EtOAc 9/1 v/v) to give the title compound as colourless oil (0.21 g).

Yield 35%.

### 3-(4-Formylphenyl)propyl nitrate

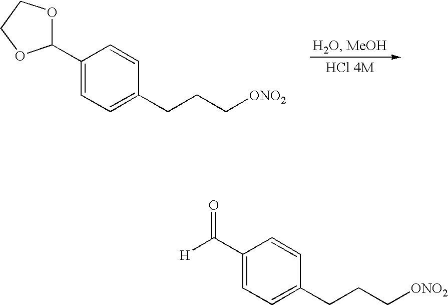

HCl 4M (5 mL) was added to a stirred solution of 3-[4-(1,3-dioxolan-2-yl)phenyl]propyl nitrate (1.0 g, 3.95 mmol) in MeOH/H₂O 1/1 (20 mL). After 30 min the reaction was completed; the mixture was extracted twice with CH₂Cl₂ (20 mL). The combined organic layers were washed with brine (20 mL), dried with MgSO₄, filtered and concentrated under reduced pressure. The crude product so obtained was purified by flash chromatography (PE/EtOAc 9/1 v/v) to give the title compound as pale yellow oil (0.75 g).

Yield 91%.

TLC: Rf=0.24 PE/EtOAc 90/10 v/v.

¹H-NMR (CDCl₃) δ 2.10 (2H, qi), 2.83 (2H, t, J=7.5 Hz), 4.46 (2H, t, J=6.6 Hz), 7.34 (2H, d, Arom), 7.84 (2H, d, Arom), 9.99 (1H, s). MS (CI) m/z 210 (M+1)⁺.

### 4-[3-(Nitrooxy)propyl]benzoic acid

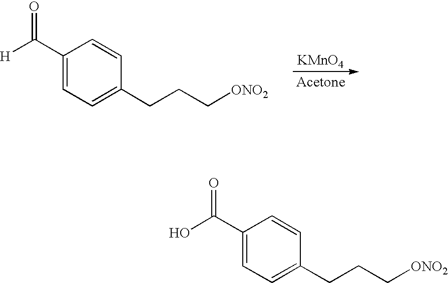

KMnO₄ (0.83 g, 5.25 mmol) was added to a solution of 3-(4-formylphenyl)propyl nitrate (0.73 g, 3.50 mmol) in acetone (20 mL), stirred at 0° C. The reaction was allowed to reach r.t. and it was completed after 1 h (TLC detection, eluent PE/EtOAc/HCOOH 70/30/0.1 v/v/v). Oxalic acid was added and the mixture was filtered and the filtrate was diluted with CH₂Cl₂ (30 mL). The organic layer was washed twice with H₂O (30 mL), then was dried with MgSO₄, filtered and concentrated under reduced pressure to give the title compound as white solid (0.70 g).

Yield 89%.

TLC: Rf=0.30 PE/EtOAc/HCOOH 80/20/0.1 v/v/v.

¹H-NMR (CDCl₃) δ 2.11 (2H, qi), 2.82 (2H, t, J=7.5 Hz), 4.46 (2H, t, J=6.3 Hz), 7.26 (2H, d, Arom), 8.06 (2H, d, Arom), 11.7 (1H, s vvbr). ¹³C-NMR (CDCl₃) δ 28.0, 31.9, 72.0, 127.6, 128.6, 130.7, 146.7, 171.9. MS (CI) m/z 226 (M+1)⁺.

### ({4-[3-(Nitrooxy)propyl]benzoyl}oxy)methyl 2-(acetyloxy)benzoate

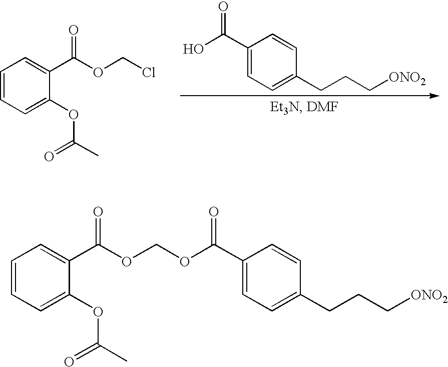

To a solution of chloromethyl 2-(acetyloxy)benzoate (0.61 g, 2.66 mmol) in dry DMF (12 mL) were added 4-[3-(nitrooxy)propyl]benzoic acid (0.60 g, 2.66 mmol), Et₃N (0.37 mL, 2.66 mmol) and catalytic amount of KI. The mixture was stirred for 8 days, then was poured in H₂O (50 mL) and extracted with Et₂O (3×50 mL). The combined organic layers were washed with NaHCO₃ 1N (2×30 mL), dried with MgSO₄, filtered and concentrated under reduced pressure. The crude product was purified by flash chromatography (PE/EtOAc 85/15 v/v) to give the title compound (0.49 g) as colourless oil.

Yield 44%.

TLC: Rf=0.36 PE/EtOAc 80/20 v/v.

¹H-NMR (CDCl₃) δ 2.07 (2H, qi), 2.35 (3H, s), 2.80 (2H, t, J=7.2 Hz), 4.44 (2H, t, J=6.3 Hz), 6.18 (2H, s), 7.10 (1H, d, Arom), 7.13-7.35 (3H, m, Arom), 7.60 (1H, t, Arom), 8.06 (2H, d, Arom), 8.10 (1H, d, Arom). ¹³C-NMR (CDCl₃) δ 21.0, 27.9, 31.8, 71.9, 79.8, 122.0, 224.0, 127.1, 128.6, 129.6, 130.2, 132.3, 134.7, 146.6, 151.1, 163.1, 165.0, 169.7. MS (CI) m/z 418 (M+1)⁺.

### EXAMPLE 13

### [(2-{[4,5-Bis(nitrooxy)pentanoyl]oxy}benzoyl)oxy]methyl 2-(acetyloxy)benzoate: compound (13)

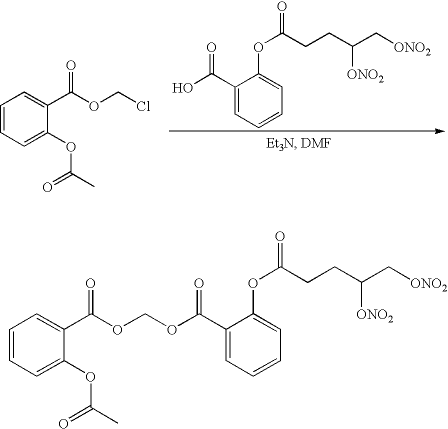

To a solution of chloromethyl 2-(acetyloxy)benzoate (0.25 g, 1.09 mmol) in dry DMF (5 mL) were added 2-{[4,5-bis(nitrooxy)pentanoyl]oxy}benzoic acid (0.38 g, 1.09 mmol), Et₃N (0.15 mL, 1.09 mmol) and catalytic amount of KI. The mixture was stirred for 8 days, then was poured in H₂O (50 mL) and extracted with Et₂O (7×10 mL). The combined organic layers were dried with MgSO₄, filtered and concentrated under reduced pressure. The crude product was purified by flash chromatography (PE/EtOAc 75/25 v/v) to give the title compound (0.15 g) as colourless oil.

Yield 25%.

TLC: Rf=0.17 PE/EtOAc 80/20 v/v.

¹H-NMR (CDCl₃) δ 2.10-2.19 (2H, m), 2.34 (3H, s), 2.81-2.87 (2H, m), 4.48-4.54 (1H, dd, AMX like system), 4.81-4.86 (1H, dd, AMX like system), 5.47-5.51 (1H, m), 6.11 (2H, s), 7.12 (2H, d, Arom), 7.31-7.38 (2H, m, Arom), 7.58-7.65 (2H, m, Arom), 8.09 (2H, t, Arom). ¹³C-NMR (CDCl₃) δ 20.9, 24.1, 29.1, 71.1, 77.9, 79.7, 121.5, 121.9, 123.8, 124.0, 126.2, 126.5, 132.2, 132.3, 134.8, 135.0, 150.9, 151.1, 162.9, 163.0, 169.6, 170.8. MS (CI) m/z 537 (M+1)⁺.

### EXAMPLE 14

### {[2-(Acetyloxy)benzoyl]oxy}methyl 2-hydroxy-4-[3-(nitrooxy)propoxy]benzoate: compound (14)

### 7-(3-Bromopropoxy)-2,2-dimethyl-4H-1,3-benzodioxin-4-one

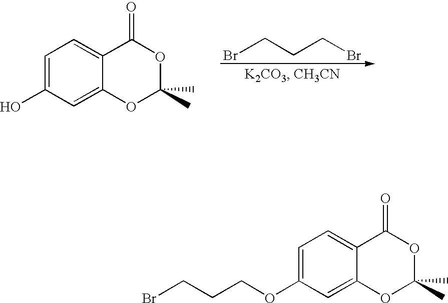

A solution of 7-hydroxy-2,2-dimethyl-4H-1,3-benzodioxin-4-one (0.50 g, 2.57 mmol; Tetrahedron 2003, 59, 6873-6887), 1,3-dibromopropane (1.3 mL, 12.85 mmol) and K₂CO₃ (0.42 g, 3.08 mmol) in CH₃CN (5 mL) was refluxed for 4.5 hours. Then the mixture was poured in H₂O (30 mL) and extracted twice with CH₂Cl₂ (15 mL); the combined organic layers were washed with brine (10 mL), dried with MgSO₄, filtered and concentrated under reduced pressure. The crude product was purified by flash chromatography (PE/EtOAc 9/1 v/v) to give the title compound (0.55 g) as pale yellow oil.

Yield 76%.

TLC: Rf=0.49 PE/EtOAc 80/20 v/v.

¹H-NMR (CDCl₃) δ 1.72 (6H, s), 2.34 (2H, qi), 3.54-3.62 (2H, m), 4.16 (2H, t, J=5.7 Hz), 6.44 (1H, s, Arom), 6.65 (1H, d, Arom), 7.85 (1H, d, Arom). ¹³C-NMR (CDCl₃) δ 25.7, 29.6, 31.9, 65.8, 101.5, 106.3, 110.5, 131.1, 157.8, 160.8, 165.3. MS (CI) m/z 315/317 (M+1)⁺.

### 3-[(2,2-Dimethyl-4-oxo-4H-1,3-benzodioxin-7-yl)oxy]propyl nitrate

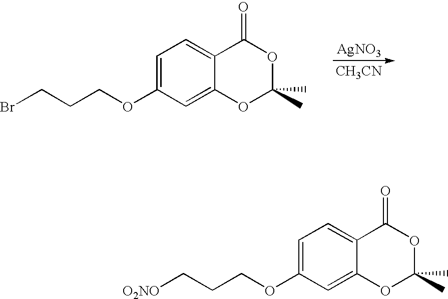

A solution of 7-(3-bromopropoxy)-2,2-dimethyl-4H-1,3-benzodioxin-4-one (0.55 g, 1.74 mmol) and AgNO₃ (0.59 g, 3.49 mmol) in CH₃CN (10 mL) was stirred at 70° C. for 3.5 h. Then brine was added to precipitate the excess of AgNO₃, the mixture was filtered through Celite and concentrated under reduced pressure. The residue was treated with EtOAc (10 mL) and H₂O (10 mL). After separation the aqueous layer was extracted twice with EtOAc (10 mL). The combined organic layers were dried with MgSO₄, filtered and concentrated under reduced pressure. The crude product was purified by flash chromatography (PE/EtOAc 90/10 v/v) to give the title compound as pale yellow oil (0.50 g).

Yield 90%.

TLC: Rf=0.15 PE/EtOAc 80/20 v/v.

¹H-NMR (CDCl₃) δ 1.73 (6H, s), 2.25 (2H, qi), 4.12 (2H, t, J=5.8 Hz), 4.67 (2H, t, J=6.0 Hz), 6.42 (1H, s, Arom), 6.64 (1H, d, Arom), 7.87 (1H, d, Arom). ¹³C-NMR (CDCl₃) δ 25.8, 26.8, 64.1, 69.5, 101.6, 106.4, 106.6, 110.4, 131.3, 157.9, 160.9, 165.0. MS (CI) m/z 298 (M+1)⁺.

### 2-Hydroxy-4-[3-(nitrooxy)propoxy]benzoic acid

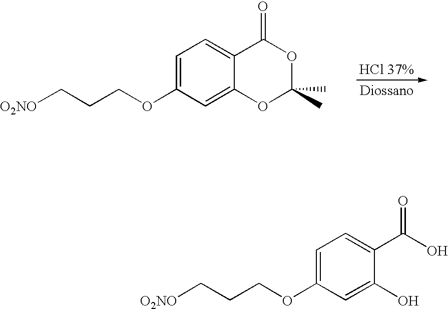

HCl 37% (0.70 mL) was added to a solution of 3-[(2,2-dimethyl-4-oxo-4H-1,3-benzodioxin-7-yl)oxy]propyl nitrate (0.50 g, 1.68 mmol) in dioxane (7 mL). The resulting mixture was heated at 60° C. for 2 hours, then was concentrated under reduced pressure. The residue was dissolved with MeOH/CH₂Cl₂ (10 mL) and concentrated under reduced pressure, the treatment was repeated 3 times to obtain the title compound as white solid (0.38 g).

Yield 92%.

TLC: Rf=0.30 PE/EtOAc/HCCOH 80/20/0.1 v/v/v.

¹H-NMR (DMSO-d₆) δ 2.15 (2H, qi), 4.12 (2H, t, J=6.0 Hz), 4.68 (2H, t, J=6.5 Hz), 6.51 (2H, m, Arom), 7.71 (1H, d, Arom). MS (CI) m/z 258 (M+1)⁺.

### {[2-(Acetyloxy)benzoyl]oxy}methyl 2-hydroxy-4-[3-(nitrooxy) propoxy]benzoate

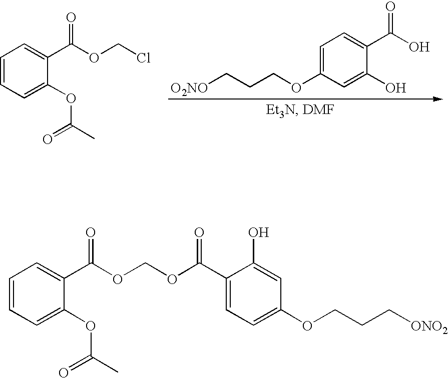

To a solution of chloromethyl 2-(acetyloxy)benzoate (0.25 g, 1.09 mmol) in dry DMF (5 mL) were added 2-hydroxy-4-[3-(nitrooxy)propoxy]benzoic acid (0.28 g, 1.09 mmol), Et₃N (0.15 mL, 1.09 mmol) and catalytic amount of KI. The mixture was stirred for 8 days, then was poured in H₂O (50 mL) and extracted with CH₂Cl₂ (5×10 mL). The combined organic layers were dried with MgSO₄, filtered and concentrated under reduced pressure. The crude product was purified by flash chromatography (PE/EtOAc 8/2 v/v) to give the title compound (0.24 g) as colourless oil.

Yield 47%.

TLC: Rf=0.10 PE/EtOAc 85/15 v/v.

¹H-NMR (CDCl₃) δ 2.22 (2H, qi), 2.35 (3H, s), 4.09 (2H, t, J=6.0 Hz), 4.65 (2H, t, J=6.0 Hz), 6.18 (2H, s), 6.43-6.45 (2H, m, Arom), 7.12 (1H, d, Arom), 7.33 (1H, t, Arom), 7.59 (1H, t, Arom), 7.80 (1H, d, Arom), 8.09 (1H, d, Arom), 10.6 (1H, s). ¹³C-NMR (CDCl₃) δ 21.0, 26.7, 63.9, 69.6, 79.5, 101.3, 104.8, 108.1, 121.9, 124.0, 126.1, 131.9, 132.2, 134.8, 151.1, 162.9, 164.3, 165.1, 168.5, 169.6. MS (CI) m/z 450 (M+1)⁺.

### EXAMPLE 15

### [(4-{[3-(Nitrooxy)propyl]sulfonyl}benzoyl)oxy]methyl 2-(acetyloxy)benzoate: compound (15)

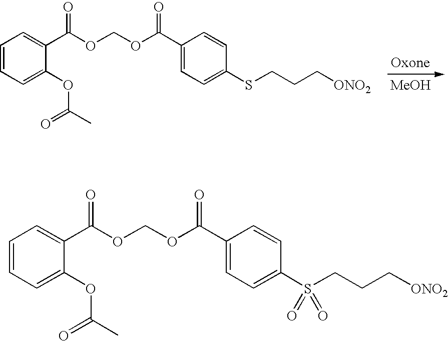

Oxone (0.4 g, 0.55 mmol) was added to a stirred solution of [(4-{[3-(nitrooxy)propyl]thio}benzoyl)oxy]methyl 2-(acetyloxy)benzoate (0.10 g, 0.22 mmol) in MeOH (3 mL) and H₂O (1 mL). 2 hours later the reaction was completed, the mixture was diluted with H₂O (10 mL) and extracted with CH₂Cl₂ (3×10 mL). The combined organic layers were dried with MgSO₄, filtered and concentrated under reduced pressure. The crude product so obtained was purified by flash chromatography (PE/EtOAc 8/2 v/v) to give the title compound as colourless oil (0.09 g).

Yield 88%.

TLC: Rf=0.50 PE/EtOAc 60/40 v/v.

¹H-NMR (CDCl₃) δ 2.18 (2H, qi), 2.36 (3H, s), 3.21 (2H, t, J=7.2 Hz), 4.55 (2H, t, J=6.0 Hz), 6.18 (2H, s), 7.12 (1H, d, Arom), 7.31 (1H, t, Arom), 7.61 (1H, t, Arom), 8.00 (2H, d, Arom), 8.10 (1H, d, Arom), 8.30 (2H, d, Arom). ¹³C-NMR (CDCl₃) δ 20.6, 21.0, 52.3, 70.2, 80.1, 121.7, 124.1, 126.2, 128.3, 131.1, 132.2, 134.0, 135.0, 143.1, 151.1, 162.9, 163.6, 169.7. MS (CI) m/z 482 (M+1)⁺.

### EXAMPLE 16

### [(4-{[2,3-Bis(nitrooxy)propyl]thio}benzoyl)oxy]methyl 2-(acetyloxy)benzoate: compound (16)

### 4-{[2,3-Bis(nitrooxy)propyl]thio}benzoic acid

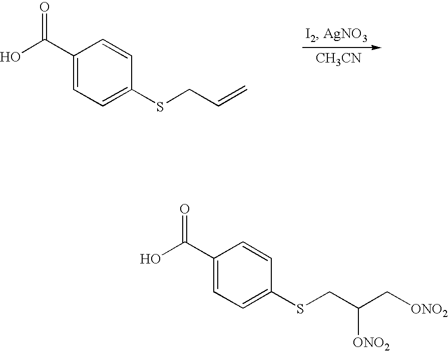

Iodine (8.2 g, 32.38 mmol) was added portion wise to a stirred solution of 4-allylthiobenzoic acid (6.30 g, 32.38 mmol; Bioorg. Med. Chem. 2002, 10, 639-656) and AgNO₃ (5.50 g, 32.38 mmol) in CH₃CN (100 mL) kept at −15° C. At the end of the addition the stirring was continued for 1 h. Then AgNO₃ (11.0 g, 64.76 mmol) was added and the mixture was heated at 70° C. for 16 h. After cooling the mixture was filtered through Celite®. The filtrate was concentrated under reduced pressure, dissolved in water (50 mL) and extracted with CH₂Cl₂ (3×100 mL). The combined organic layers were washed with brine (50 mL), dried with MgSO₄, filtered and concentrated under reduced pressure. The crude product was purified by flash chromatography (PE/EtOAc/HCOOH 80/20/0.1 v/v/v) to give the title compound as white solid (6.1 g).

Yield 60%.

TLC: Rf=0.26 PE/EtOAc/HCOOH 80/20/0.1 v/v/v.

¹H-NMR (DMSO-d₆) δ 3.50-3.63 (2H, m), 4.77-5.05 (2H, m, AMX like system), 5.51-5.58 (1H, m), 7.53 (2H, d, Arom), 7.87 (2H, d, Arom), 13.00 (1H, s). ¹³C-NMR (DMSO-d₆) δ 30.0, 70.8, 78.1, 127.3, 128.2, 129.5, 138.6, 166.7. MS (CI) m/z 319 (M+1)⁺.

### [(4-{[2,3-Bis(nitrooxy)propyl]thio}benzoyl)oxy]methyl 2-(acetyloxy)benzoate

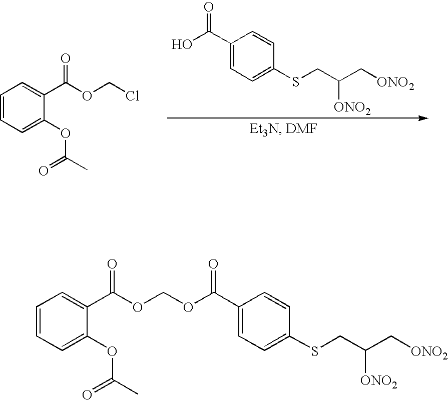

To a solution of chloromethyl 2-(acetyloxy)benzoate (0.33 g, 1.44 mmol) in dry DMF (10 mL) were added 4-{[2,3-bis(nitrooxy)propyl]thio}benzoic acid (0.46 g, 1.44 mmol), Et₃N (0.20 mL, 1.44 mmol) and catalytic amount of KI. The mixture was stirred for 9 days, then was poured in H₂O (50 mL) and extracted with Et₂O (3×50 mL). The combined organic layers were dried with MgSO₄, filtered and concentrated under reduced pressure. The crude product was purified by flash chromatography (PE/EtOAc 8/2 v/v) to give the title compound (0.32 g) as colourless oil.

Yield 37%.

TLC: Rf=0.24 PE/EtOAc 80/20 v/v.

¹H-NMR (CDCl₃) δ 2.35 (3H, s), 3.20-3.28 (1H, dd, AMX like system), 3.35-3.42 (1H, dd, AMX like system), 4.63-4.69 (1H, dd, AMX like system), 4.86-4.91 (1H, dd, AMX like system), 5.28-5.36 (1H, m), 6.19 (2H, s), 7.12 (1H, d, Arom), 7.33 (1H, t, Arom), 7.37 (2H, d, Arom), 7.60 (1H, t, Arom), 8.04 (2H, d, Arom), 8.07 (1H, d, Arom). ¹³C-NMR (CDCl₃) δ 21.0, 31.4, 69.3, 77.2, 79.9, 121.9, 124.0, 126.2, 127.4, 128.3, 130.6, 132.3, 134.8, 141.1, 151.1, 163.0, 164.5, 169.7. MS (CI) m/z 511 (M+1)⁺.

### EXAMPLE 17

### [(4-{[2,3-Bis(nitrooxy)propyl]sulfonyl}benzoyl)oxy]methyl 2-(acetyloxy)benzoate: compound (17)

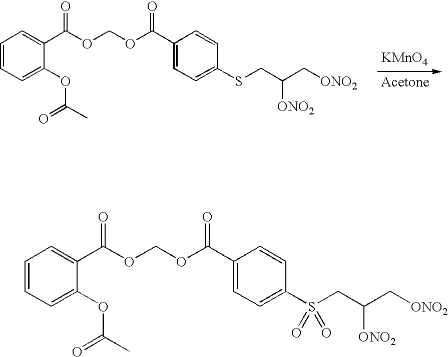

KMnO₄ (0.09 g, 0.58 mmol) was added to a solution of [(4-{[2,3-bis(nitrooxy)propyl]thio}benzoyl)oxy]methyl 2-(acetyloxy)benzoate (0.20 g, 0.39 mmol) in acetone (10 mL), stirred at 0° C. The reaction was allowed to reach r.t. and it was completed after 1 h (TLC detection, eluent PE/EtOAc 8/2 v/v). Oxalic acid was added and the mixture was filtered and the filtrate was diluted with CH₂Cl₂ (15 mL). The organic layer was washed twice with H₂O (10 mL), then was dried with MgSO₄, filtered and concentrated under reduced pressure. The crude product so obtained was purified by column chromatography (PE/EtOAc 8/2 v/v) to give the title compound as white solid (0.20 g).

Yield 95%.

TLC: Rf=0.58 PE/EtOAc 60/40 v/v.

¹H-NMR (CDCl₃) δ 2.36 (3H, s), 3.50-3.60 (2H, m), 4.62-4.68 (1H, dd, AMX like system), 4.93-4.98 (1H, dd, AMX like system), 5.70-5.75 (1H, m), 6.22 (2H, s), 7.13 (1H, d, Arom), 7.34 (1H, t, Arom), 7.63 (1H, t, Arom), 8.01 (2H, d, Arom), 8.09 (1H, d, Arom), 8.33 (2H, d, Arom). ¹³C-NMR (CDCl₃) δ 21.0, 54.7, 69.8, 72.8, 79.9, 121.7, 124.1, 126.2, 128.2, 131.3, 132.2, 134.5, 135.0, 143.0, 151.2, 162.9, 163.5, 169.7. MS (CI) m/z 543 (M+1)⁺.

### EXAMPLE 18

### [(4-{[3-(Nitrooxy)propyl]sulfonyl}benzoyl)oxy]methyl 2-(acetyloxy)benzoate: compound (18)

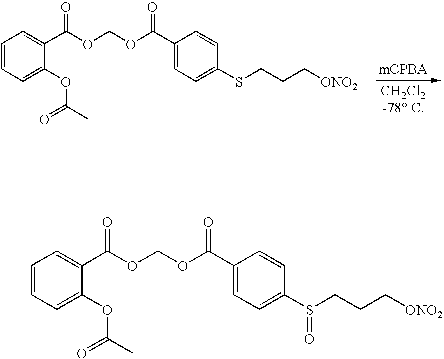

A solution of mCPBA 70% (0.25 g, 1.0 mmol) in dry CH₂Cl₂ (7 mL) was slowly added to a solution of [(4-{[3-(nitrooxy)propyl]thio}benzoyl)oxy]methyl 2-(acetyloxy)benzoate (0.45 g, 1.0 mmol) in dry CH₂Cl₂ (7 mL), stirred at −78° C. At the end of the addition the reaction was completed. The mixture was poured in Na₂SO₃ 10% (50 mL), the layers separated and the aqueous layer was extracted twice with Et₂O (50 mL). The organic layers were dried with MgSO₄, filtered and concentrated under reduced pressure. The crude product so obtained was purified by flash chromatography (PE/EtOAc 6/4 v/v) to give the title compound as colourless oil (0.34 g).

Yield 73%.

TLC: Rf=0.15 PE/EtOAc 60/40 v/v.

¹H-NMR (CDCl₃) δ 1.97-2.05 (1H, m), 2.23-2-33 (1H, m), 2.37 (3H, s), 2.76-2.85 (1H, m), 2.97-3.07 (1H, m), 4.52-4.57 (2H, m), 6.21 (2H, s), 7.13 (1H, d, Arom), 7.33 (1H, t, Arom), 7.59 (1H, t, Arom), 7.69 (2H, d, Arom), 8.11 (1H, d, Arom), 8.24 (2H, d, Arom). ¹³C-NMR (CDCl₃) δ 19.7, 21.0, 52.3, 71.1, 79.9, 121.8, 124.1, 124.1, 126.2, 131.0, 131.6, 132.5, 134.9, 149.1, 151.2, 162.9, 164.2, 169.7. MS (CI) m/z 466 (M+1)⁺.

### EXAMPLE 19

### [(4-{[(3-Methyl-furoxan-4-yl)methyl]thio}benzoyl)oxy]methyl 2-(acetyloxy)benzoate: compound (20)

### 4-{[(3-Methyl-furoxan-4-yl)methyl]thio}benzoic acid

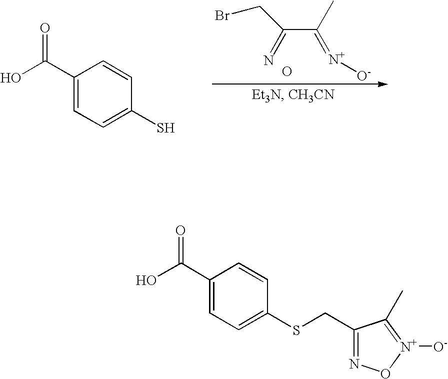

To a sospension of 4-mercaptobenzoic acid 90% (1.0 g, 5.34 mmol) in CH₃CN (10 mL), stirred at 0° C., 4-bromomethyl-3-methyl furoxan (1.13 g, 5.84 mmol; J. Med. Chem. 1998, 41, 5393-5401) and Et₃N (1.63 mL, 11.67 mmol) were added. After 2 hours the reaction was completed. The mixture was poured in HCl 1M (20 mL) and extracted with EtOAc (3×20 mL); the combined organic layers were dried with MgSO₄, filtered and concentrated under reduced pressure. The crude product was treated with iPr₂O/MeOH to give the title compound (1.41 g) as white solid.

Yield 90%.

TLC: Rf=0.31 PE/EtOAc/HCOOH 60/40/0.1 v/v/v.

¹H-NMR (DMSO-d₆) δ 2.23 (3H, s), 4.25 (2H, s), 7.43 (2H, d, Arom), 7.95 (2H, d, Arom). ¹³C-NMR (CDCl₃) δ 7.8, 27.3, 112.4, 128.5, 129.4, 130.0, 139.1, 154.7, 167.4. MS (CI) m/z 267 (M+1)⁺.

### [(4-{[(3-Methyl-furoxan-4-yl)methyl]thio}benzoyl)oxy]methyl 2-(acetyloxy)benzoate

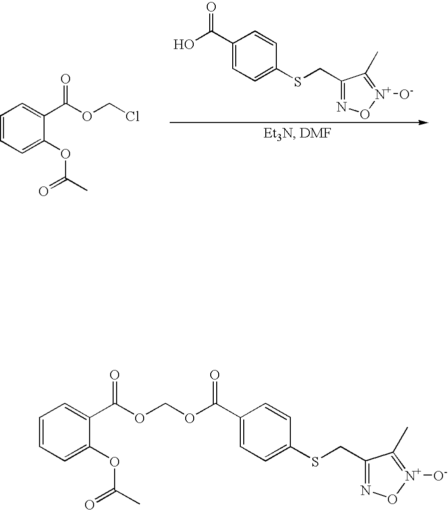

To a solution of chloromethyl 2-(acetyloxy)benzoate (0.90 g, 3.94 mmol) in dry DMF (10 mL) were added 4-{[(3-methyl-furoxan-4-yl)methyl]thio}benzoic acid (1.05 g, 3.94 mmol), Et₃N (0.55 mL, 3.94 mmol) and catalytic amount of KI. The mixture was stirred for 7 days, then was poured in H₂O (50 mL) and extracted with CH₂Cl₂ (20 mL). The organic layer was dried with MgSO₄, filtered and concentrated under reduced pressure. The crude product was purified by flash chromatography (CH₂Cl₂/EtOAc 8/2 v/v) to give the title compound (0.90 g) as white solid.

Yield 50%.

m.p. 96-97° C. (from iPr₂O)

TLC: Rf=0.12 PE/EtOAc 80/20 v/v.

¹H-NMR (CDCl₃) δ 2.21 (3H, s), 2.35 (3H, s), 4.16 (2H, s), 6.18 (2H, s), 7.12 (1H, d, Arom), 7.26 (1H, t, Arom), 7.35 (2H, d, Arom), 7.58 (1H, t, Arom), 8.08 (2H, d, Arom), 8.10 (1H, d, Arom). ¹³C-NMR (CDCl₃) δ 7.95, 21.0, 27.4, 80.2, 112.2, 121.9, 124.0, 126.2, 127.3, 128.4, 130.8, 132.2, 134.7, 140.6, 151.1, 154.1, 163.0, 164.5, 169.7. MS (CI) m/z 459 (M+1)⁺.

### EXAMPLE 20

### [(4-{[(3-Aminocarbonyl-furoxan-4-yl)methyl]thio}benzoyl) oxy]methyl 2-(acetyloxy)benzoate: compound (21)

### 4-{[3-(Aminocarbonyl)-furoxan-4-yl]methyl}thio)benzoic acid

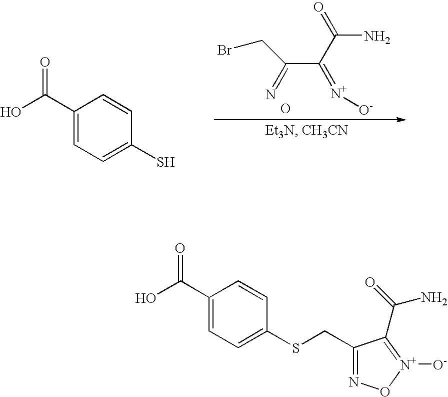

To a sospension of 4-mercaptobenzoic acid 90% (1.16 g, 6.75 mmol) in CH₃CN (10 mL), stirred at 0° C., 4-bromomethyl-3-aminocarbonyl furoxan (1.5 g, 6.75 mmol; J. Med. Chem. 1998, 41, 5393-5401) and Et₃N (1.9 mL, 13.5 mmol) were added. After 1 hours the reaction was completed. The mixture was poured in HCl 1M (20 mL) and extracted with CH₂Cl₂ (3×20 mL); the combined organic layers were dried with MgSO₄, filtered and concentrated under reduced pressure. The crude product was treated with iPr₂O/CH₂Cl₂ to give the title compound (1.69 g) as white solid.

Yield 85%.

TLC: Rf=0.16 PE/EtOAc/HCOOH 80/20/0.1 v/v/v.

¹H-NMR (CD₃OD) δ 4.53 (2H, s), 7.50 (2H, d, Arom), 7.97 (2H, d, Arom). ¹³C-NMR (CD₃OD+DMSO-d₆) δ 27.9, 126.8, 128.6, 129.3, 130.7, 131.1, 141.8, 156.9, 167.9. MS (CI) m/z 296 (M+1)⁺.

### [(4-{[(3-Aminocarbonyl-furoxan-4-yl)methyl]thio}benzoyl) oxy]methyl 2-(acetyloxy)benzoate

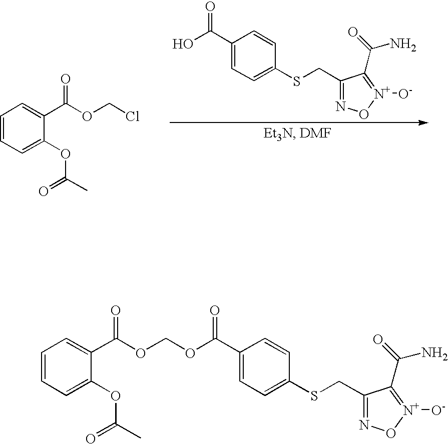

To a solution of chloromethyl 2-(acetyloxy)benzoate (0.90 g, 3.94 mmol) in dry DMF (10 mL) were added 4-{[3-(aminocarbonyl)-furoxan-4-yl]methyl}thio)benzoic acid (1.16 g, 3.94 mmol), Et₃N (0.55 mL, 3.94 mmol) and catalytic amount of KI. The mixture was stirred for 9 days, then was poured in H₂O (50 mL) and extracted with CH₂Cl₂ (5×20 mL). The organic layer was dried with MgSO₄, filtered and concentrated under reduced pressure. The crude product was purified by flash chromatography (CH₂Cl₂/EtOAc 9/1 v/v) to give the title compound (0.41 g) as white solid.

Yield 46%.

m.p. 149-151° C. (from iPr₂O/iPrOH).

TLC: Rf=0.33 PE/EtOAc 60/40 v/v.

¹H-NMR (CDCl₃) δ 2.35 (3H, s), 4.49 (2H, s), 5.96 (1H, s br), 6.18 (2H, s), 7.12 (1H, d, Arom), 7.30 (1H, t, Arom), 7.44 (2H, d, Arom), 7.51 (1H, s br), 7.58 (1H, t, Arom), 8.05 (2H, d, Arom), 8.10 (1H, d, Arom). ¹³C-NMR (CDCl₃) δ 21.0, 27.7, 79.8, 110.0, 122.0, 124.0, 125.9, 126.9, 128.0, 130.8, 132.3, 134.7, 141.9, 151.1, 155.5, 163.0, 164.7, 169.7. MS (CI) m/z 488 (M+1)⁺.

### EXAMPLE 21

### [(4-{[(3-Cyano-furoxan-4-yl)methyl]thio}benzoyl)oxy]methyl 2-(acetyloxy)benzoate: compound (22)

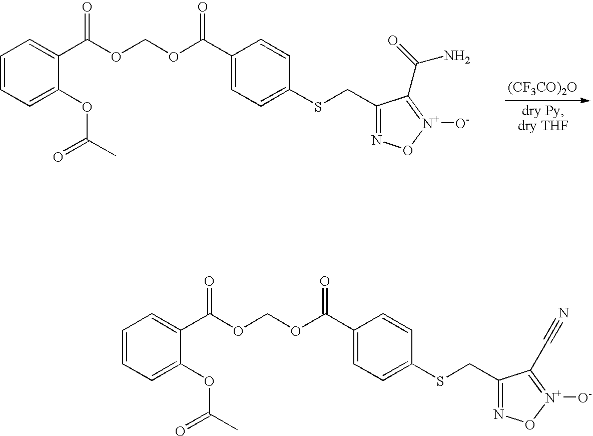

Trifluoroacetic anhydride (0.10 mL, 0.55 mmol) was slowly added to a stirred solution, kept under inert atmosphere at 0° C., of [(4-{[(3-aminocarbonyl-furoxan-4-yl)methyl]thio}benzoyl)oxy]methyl 2-(acetyloxy)benzoate (0.14 g, 0.29 mmol) and dry pyridine (0.05 mL, 0.58 mmol) in dry THF (6 mL). After 20 min the reaction was completed. The mixture was poured in H₂O (10 mL) and extracted twice with Et₂O (10 mL). the organic layers were washed with HCl 0.5 M (10 mL), dried with MgSO₄, filtered and concentrated under reduced pressure. The crude product so obtained was treated with PE/MeOH to give the title compound as white solid (0.12 g).

Yield 86%.

m.p. 98.5-101.5° C. (from iPr₂O/iPrOH).

TLC: Rf=0.21 PE/EtOAc 80/20 v/v.

¹H-NMR (CDCl₂) δ 2.35 (3H, s), 4.24 (2H, s), 6.18 (2H, s), 7.12 (1H, d, Arom), 7.32 (1H, t, Arom), 7.45 (2H, d, Arom), 7.60 (1H, t, Arom), 8.07 (2H, d, Arom), 8.10 (1H, d, Arom). ¹³C-NMR (CDCl₂) δ 21.0, 27.4, 79.9, 104.9, 121.9, 124.0, 126.2, 128.0, 129.0, 129.5, 131.0, 132.2, 134.8, 139.2, 151.1, 153.7, 163.0, 164.4, 169.7. MS (CI) m/z 470 (M+1)⁺.

### EXAMPLE 22

### {[2-(Acetyloxy)benzoyl]oxy}methyl 6-[(nitrooxy)methyl]pyridine-2-carboxylate: compound (23)

### {[2-(Acetyloxy)benzoyl]oxy}methyl 6-(hydroxymethyl)pyridine-2-carboxylate

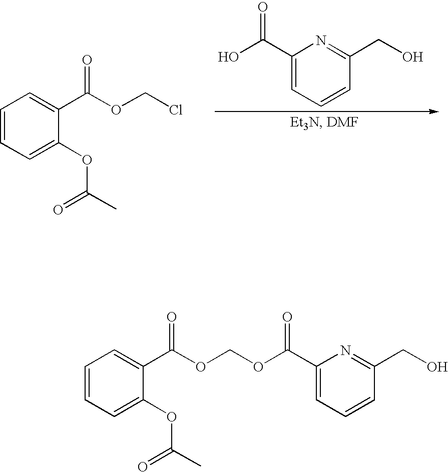

To a solution of chloromethyl 2-(acetyloxy)benzoate (0.50 g, 2.18 mmol) in dry DMF (10 mL) were added 6-(hydroxymethyl)pyridine-2-carboxylic acid (0.33 g, 2.18 mmol; J. Med. Chem. 2006, 49, 2628-2639) and Et₃N (0.30 mL, 2.18 mmol). The mixture was stirred for 6 days, then was poured in H₂O (50 mL) and extracted with Et₂O (3×10 mL). The organic layers were dried with MgSO₄, filtered and concentrated under reduced pressure. The crude product was purified by flash chromatography (CH₂Cl₂/EtOAc 9/1 v/v) to give the title compound (0.06 g) as colourless oil.

Yield 25%.

TLC: Rf=0.20 CH₂Cl₂/EtOAc 95/5 v/v.

¹H-NMR (CD₃OD) δ 2.29 (3H, s), 4.76 (2H, s), 6.24 (2H, s), 7.18 (1H, d, Arom), 7.39 (1H, t, Arom), 7.66 (1H, t, Arom), 7.80 (1H, d, Arom), 7.99-8.09 (3H, m, Arom). ¹³C-NMR (CD₃OD) 21.0, 65.3, 81.8, 118.1, 120.0, 124.6, 125.3, 127.4, 131.6, 132.9, 134.9, 139.6, 148.0, 163.5, 163.8. MS (CI) m/z 346 (M+1)⁺.

### {[2-(Acetyloxy)benzoyl]oxy}methyl 6-[(nitrooxy)methyl]pyridine-2-carboxylate

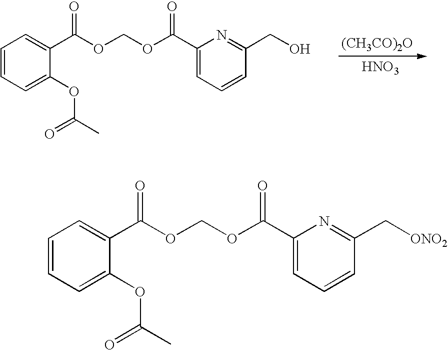

A solution of {[2-(acetyloxy)benzoyl]oxy}methyl 6-(hydroxymethyl)pyridine-2-carboxylate (0.10 g, 0.29 mmol) in (CH₃CO)₂O (0.30 mL) was slowly added to a mixture of HNO₃ 65% (0.10 mL) and (CH₃CO)₂O (0.20 mL), stirred at 0° C. Then the reaction mixture was allowed to reach room temperature and the stirring was continued for 2 hours. The mixture was poured into H₂O (10 mL) and extracted with CH₂Cl₂ (5×5 mL). The organic layers were dried with MgSO₄, filtered and concentrated under reduced pressure. The crude product so obtained was purified by flash chromatography (CH₂Cl₂/EtOAc 95/5 v/v) to give the title compound as colourless oil that became solid on standing.

Yield 50%.

TLC: Rf=0.74 CH₂Cl₂/EtOAc 90/10 v/v.

¹H-NMR (CDCl₃) δ 2.37 (3H, s), 5.67 (2H, s), 6.26 (2H, s), 7.12 (1H, d, Arom), 7.34 (1H, t, Arom), 7.58-7.63 (2H, m, Arom), 7.93 (1H, t, Arom), 8.09-8.17 (2H, m, Arom). ¹³C-NMR (CDCl₃) δ 21.0, 73.9, 80.5, 121.8, 124.0, 125.4, 125.6, 126.2, 132.3, 134.8, 138.4, 147.0, 151.2, 153.8, 162.8, 163.2, 169.7. MS (CI) m/z 391 (M+1)⁺.

### EXAMPLE 23

### 1-{[4-([3-(nitrooxy)propyl]thio}benzoyl)oxy]ethyl 2-(acetyloxy)Benzoate: compound (24)

### 1-(chloroethyl) 2-(acetyloxy)benzoate

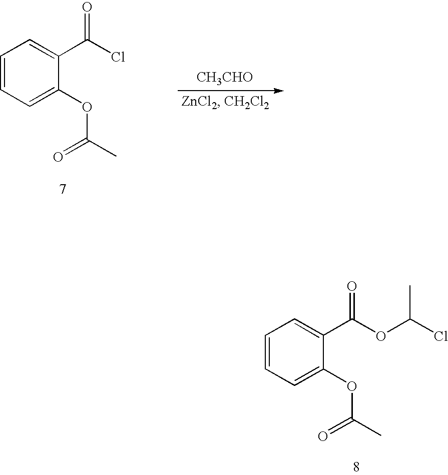

To a solution of 2-(chlorocarbonyl)phenyl acetate (10.0 g, 50.35 mmol) in dry CH₂Cl₂ (100 mL), kept under inert atmosphere, ZnCl₂ (0.14 g, 1.01 mmol) was added. After 15 min the reaction mixture was cooled at −15° C. and a solution of CH₃CHO (2.8 mL, 50.35 mmol) in dry CH₂Cl₂ (30 mL) was slowly added. Then the reaction was allowed to reach room temperature and stirred for 18 hours. The mixture was washed with H₂O (100 mL) and a saturated solution of NaHCO₃ (100 mL), dried with MgSO₄, filtered and concentrated under reduced pressure. The crude product was purified by flash chromatography (PE/EtOAc 9/1 v/v) to give the title compound (7.84 g) as a colourless oil.

Yield 64%.

TLC: Rf=0.35 PE/EtOAc 90/10 v/v.

¹H-NMR (CDCl₃) δ 1.89 (3H, d), 2.37 (3H, s), 6.73 (1H, q), 7.13 (1H, d, Arom), 7.34 (1H, t, Arom), 7.60 (1H, t, Arom), 8.04 (1H, d, Arom). ¹³C-NMR (CDCl₃) δ 21.0, 25.3, 81.1, 122.1, 124.0, 126.1, 132.0, 134.7, 151.0, 162.0, 169.5. MS (CI) m/z 242/244 (M+1)⁺.

### 1-[(4-{[3-(nitrooxy)propyl]thio}benzoyl)oxy]ethyl 2-(acetyloxy)benzoate

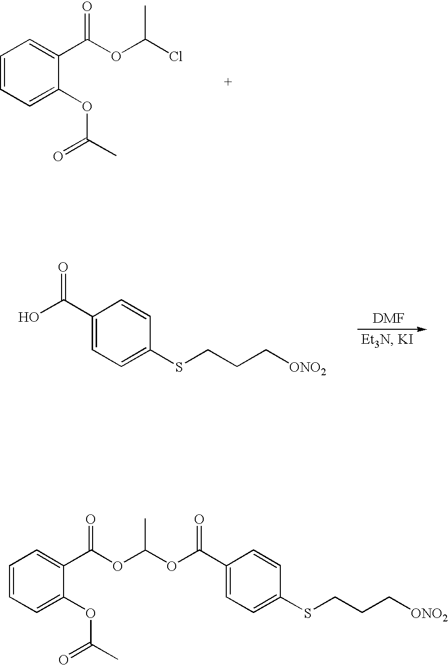

To a solution of 1-(chloroethyl) 2-(acetyloxy)benzoate (0.50 g, 2.06 mmol) in dry DMF (10 mL) were added 4-{[3-(nitrooxy)propyl]thio}benzoic acid (0.53 g, 2.06 mmol), Et₃N (0.28 mL, 2.06 mmol) and catalytic amount of KI. The mixture was stirred for 10 days, then was poured in H₂O (30 mL) and extracted with Et₂O (4×30 mL). The combined organic layers were washed with NaHCO₃ 1N (2×30 mL), dried with MgSO₄, filtered and concentrated under reduced pressure. The crude product was purified by flash chromatography (PE/EtOAc 9/1 v/v) to give the title compound (0.11 g) as colourless oil.

Yield 11%.

TLC: Rf=0.20 PE/EtOAc 90/10 v/v.

¹H-NMR (CDCl₃) δ 1.72 (3H, d), 2.10 (2H, qi), 2.30 (3H, s), 3.10 (2H, t), 4.58 (2H, t), 7.11 (1H, d, Arom), 7.29-7.34 (4H, m, Arom), 7.58 (1H, t, Arom), 7.97 (2H, d, Arom), 8.04 (1H, d, Arom), ¹³C-NMR (CDCl₃) δ 19.8, 21.0, 26.1, 28.3, 71.0, 89.6, 122.5, 123.9, 126.1, 126.4, 127.0, 130.5, 132.0, 134.4, 143.3, 150.9, 162.4, 164.0, 169.5. MS (CI) m/z 463 (M+1)⁺.

### Alternative Procedure

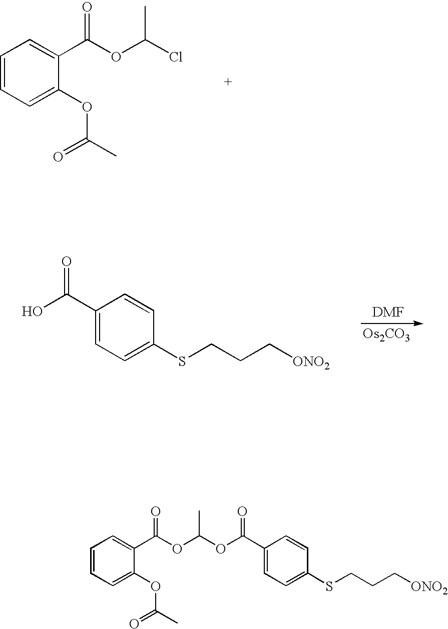

To a solution of 1-(chloroethyl) 2-(acetyloxy)benzoate (0.50 g, 2.19 mmol) in dry DMF (10 mL) were added 4-{[3-(nitrooxy)propyl]thio}benzoic acid (0.56 g, 2.19 mmol) and cesium carbonate (0.34 g, 1.1 mmol). The mixture was stirred for 4 days then was poured in H₂O (30 mL) and extracted with Et₂O (3×20 mL). the combined organic layers were washed twice with a saturated solution of NaHCO₃ (20 mL), dried with MgSO₄, filtered and concentrated under reduced pressure. The crude product was purified by flash chromatography (PE/EtOAc 9/1 v/v) to give the title compound (0.21 g) as a colourless oil.

Yield 22%.

### Hydrolysis Experiments

### Hydrolysis in Acidic Medium (pH 1) and in Phosphate Buffer (pH 7.4

A solution of each compound of the invention (10 mM) in acetonitrile was added to a HCl 0.1M or to a phosphate buffer 50 mM pH=7.4, containing, when necessary, 10-20% of acetonitrile as cosolvent. The final concentration of the compound was 250 μM. Resulting solution was kept at 37±0.5° C. and at appropriate time intervals a 20 μL aliquote of reaction solution was analysed by RP-HPLC.

### Hydrolysis in Human Serum

A solution of each compound of the invention (10 mM) in acetonitrile was added to human serum (from human male AB plasma, Sigma) preheated at 37° C., the final concentration of the compound was 250 μM. Resulting solution were incubated at 37±0.5° C. and at appropriate time intervals 500 μL of reaction mixture was withdrawn and added to 750 μL of acetonitrile containing 0.1% trifluoroacetic acid in order to deproteinize the serum. Sample was sonicated, vortexed and then centrifuged for 10′ at 2150 g. The clear supernatant was filtered by 0.45 μm PTFE filters (Alltech) and analysed by RP-HPLC.

Analyses were carried out with a HP 1100 chromatograph system (Agilent Technologies, Palo Alto, Calif., USA) equipped with a quaternary pump (model G1311A), a membrane degasser (G1379A), a diode-array detector (DAD) (model G1315B) integrated in the HP1100 system. Data analysis was done using a HP ChemStation system (Agilent Technologies). The analytical column was a Nucleosil 100-5018 Nautilus (250×4.6 mm, 5 μm particle size) (Macherey-Nagel). The mobile phase consisting of acetonitrile/water (55/45) with 0.1% trifluoroacetic acid and the flow-rate was 1.2 mL/min. The injection volume was 20 μL (Rheodyne, Cotati, Calif.). The column effluent was monitored at 240 nm (for salicylic acid) and at 226 nm (for all the other products) and referenced against a 360 nm wavelength. Quantitation was done by comparison of peak areas with standards chromatographed under the same conditions. The hydrolysis followed first-order kinetics. The observed pseudo-first-order rate constants (k_{obs}) were calculated from the slopes of linear plots of the natural logarithms of percent remaining products against time and the corresponding half-lives (t_{1/2}) were obtained from

t_{1/2}=0.693/k_{obs}

The results are reported in Table 1.

The compounds of the invention are stable in acid media and release aspirin when incubated in human serum.

TABLE 1
human serum and buffered solutions stability

| | | | Stability in buffered solutions | |
|---|---|---|---|---|
| | Human serum stability | | pH 1 | pH 7.4 |
| | t_{1/2} | % max of | % unchanged | % unchanged |
| Compound | (min) | ASA released | after 6 h | after 6 h |
| Aspirin | 63 | — | — | — |
| (1) | 3.4 | 34% (between | >98% | 100% |
| | | 10 and 20 min) | | |
| (2) | 3.0 | 40% (between | >98% | >98% |
| | | 10 and 20 min) | | |
| (3) | 5.4 | 24% (between | >98% | >98% |
| | | 10 and 20 min) | | |
| (6) | 2.7 | 10% (between | >98% | 100% |
| | | 10 and 20 min) | | |
| (8) | 2.5 | 8% (between | >98% | 100% |
| | | 10 and 20 min) | | |
| (9) | 1.5 | 16% (between | >98% | >98% |
| | | 10 and 20 min) | | |
| (11) | 2.0 | 64% (between | >98% | >98% |
| | | 10 and 20 min) | | |
| (15) | 1.0 | 60% (between | >98% | 70% |
| | | 5 and 10 min) | | (t_{1/2} ≈ 12 h) |
| (16) | 4.8 | 42% (between | >98% | >98% |
| | | 10 and 20 min) | | |
| (24) | 4.2 | 29% | >98% | >98% |

### Vasodilating Activity

Thoracic aortas were isolated from male Wistar rats weighing 180-200 g. The endothelium was removed and the vessels were helically cut: three strips were obtained from each aorta. The tissues were mounted under 1.0 g tension in organ baths containing 30 ml of Krebs-bicarbonate buffer with the following composition (mM): NaCl 111.2, KCl 5.0, CaCl₂ 2.5, MgSO₄ 1.2, KH₂PO₄ 1.0, NaHCO₃ 12.0, glucose 11.1, maintained at 37° C. and gassed with 95% O₂-5% CO₂ (pH=7.4). The aortic strips were allowed to equilibrate for 120 min and then contracted with 1 μM L-phenylephrine. When the response to the agonist reached a plateau, cumulative concentrations of the vasodilating agent were added.

All the compounds of the invention were capable to relax precontracted rat aorta strips in a concentration dependent manner. Vasodilating potencies expressed as EC₅₀, calculated by a linear regression analysis, are reported in Table 2.

When the vasodilator experiments were repeated in the presence of 1 μM ODQ (1H-[1,2,4]oxadiazolo[4,3-a]quinoxalin-1-one), a well known inhibitor of the soluble guanylate cyclase (sGC), a decrease in the vasodilator potencies was observed.

TABLE 2
vasodilating activity

| Compound | EC₅₀ ± SE μM |
|---|---|
| Aspirin | no effect |
| (1) | 0.052 ± 0.007 |
| (2) | 0.017 ± 0.003 |
| (3) | 0.041 ± 0.007 |
| (4) | 1.9 ± 0.5 |
| (5) | 1.2 ± 0.2 |
| (6) | 0.39 ± 0.06 |
| (7) | 1.6 ± 0.2 |
| (8) | 0.52 ± 0.09 |
| (9) | 0.30 ± 0.06 |
| (10) | 0.27 ± 0.05 |
| (12) | 0.075 ± 0.014 |
| (13) | 0.082 ± 0.014 |
| (14) | 0.0080 ± 0.0011 |

### Inhibition of Platelet Aggregation In Vitro

The ability of aspirin nitroderivatives to inhibit platelet aggregation was evaluated in vitro in human platelets.

Venous blood samples were obtained from healthy volunteers who had not taken any drug for at last two weeks. Platelet rich plasma (PRP) is prepared by centrifugation of citrated blood at 200 g for 20 minutes. Aliquots (500 μL) of PRP were added into aggregometer (Chrono-log modello 4902D) cuvettes and aggregation is recorded as increased light transmission under continuous stirring (1000 rpm) at 37° C. for 10 minutes after addition of the stimulus (collagen). Collagen (1.0 μg/mL) is used as platelet activator in PRP. The inhibitory activity of the compounds is tested by addition of drug to PRP 10 or 30 min before addition of the stimulus. Drug vehicle (0.5% DMSO) added to PRP did not affect platelet function in control samples.

The antiaggregatory activity of the compounds of the invention is evaluated as % inhibition of platelet aggregation compared to control samples. The nitroderivatives were able to inhibit platelet aggregation and resulted more potent than aspirin.

IC₅₀, values calculated by non-linear regression analysis, are reported in Table 3.

TABLE 3

Platelet Aggregation IC₅₀ (μM) (collagen 95%)

| Compound | 10 min | 30 min |
|---|---|---|
| Aspirin | 59 | 21 |
| (12) | 41 | 14 |
| (15) | 34 | 17 |

1. A compound of general formula (I) and pharmaceutically acceptable salts or stereoisomers thereof: 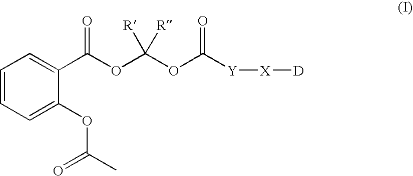
wherein:
R′ and R″ are independently H, straight or branched C₁-C₆ alkyl or when taken together R′ and R″ form a cycloalkyl from 3 to 7 carbon atoms;
Y is a bivalent radical having the following meanings:
a) straight or branched C₁-C₁₀ alkylene, optionally substituted with one or more of the substituents selected from the group consisting of: halogen atoms, hydroxy, —ONO₂, —OC(O)(C₁-C₁₀ alkyl)-ONO₂ and —O(C₁-C₁₀ alkyl)-ONO₂;
b) 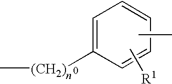
wherein R¹ is H, —COOH, —OH, CH₃ or Halogen, n⁰ is an integer from 0 to 10;
wherein the X moiety is not linked to —(CH₂)ₙ⁰;
c) 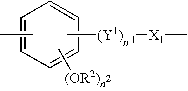
wherein:
n¹ is an integer from 0 to 1;
n² is an integer from 0 to 2;
Y¹ is —CH₂—CH₂— or —CH═CH—(CH₂)n²—;
X₁=—OCO— or —COO— and R² is H or CH₃;
wherein the X moiety is linked to X₁;
d) 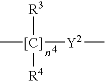
wherein:
n⁴ is an integer from 0 to 10;
R³ and R⁴ are the same or different, and are H or straight or branched C₁-C₆ alkyl;
wherein the X moiety is linked to Y²;
Y² is an heterocyclic saturated, unsaturated or aromatic 5 or 6 members ring, containing one or more heteroatoms selected from nitrogen, oxygen, sulfur, and is selected from the group consisting of: 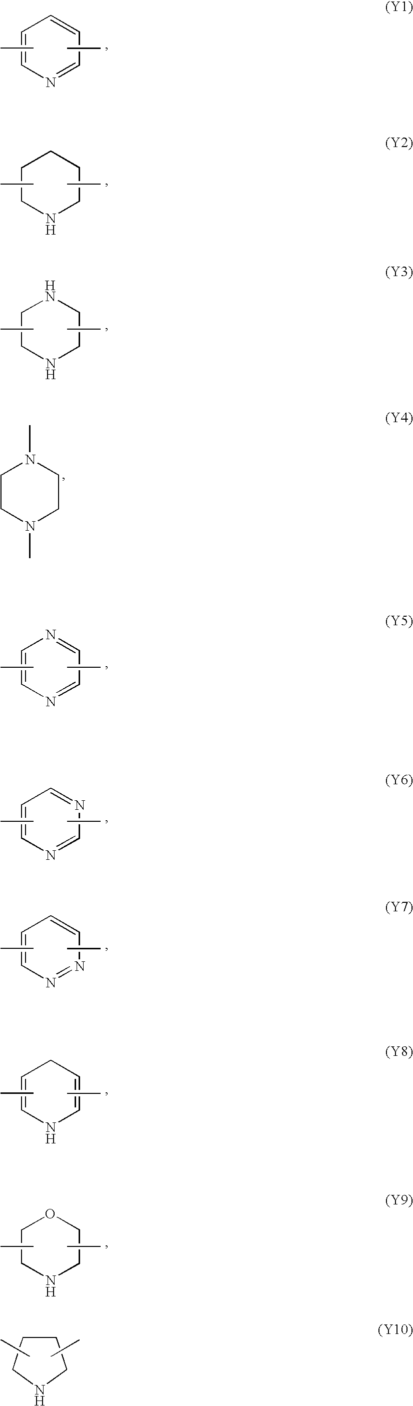 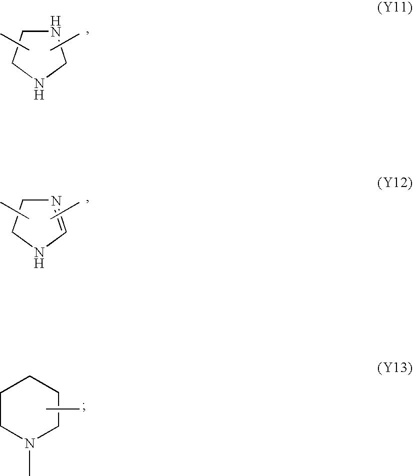
X is a moiety selected from the group consisting of: C₁-C₁₀ alkylene, —O—C₁-C₁₀ alkylene, alkylene, —S(O)—C₁-C₁₀ alkylene and —S(O)₂—C₁-C₁₀ alkylene, optionally containing solubilising groups such as —OH, —NH₂, —COOH;
D=—ONO₂, alkyl substituted with one or more —ONO₂ groups, preferably —CH(ONO₂)CH₂ONO₂ or 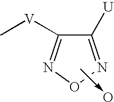
wherein V is —CH₂, —O—, —S— or —NH—; U is C₁-C₁₀ alkyl, optionally substituted with —OH or —NH₂, aryl, C₁-C₁₀ alkoxy, aryloxy, C₁-C₁₀ thioalkyl, thioaryl, halogen, di-C₁-C₁₀ (alkylamino), diarylamino, aryl C₁-C₁₀ (alkylamino), (alkylsulphoxy), arylsulphoxy, (alkylsulphone), arylsulphone, —CN, —NO₂, —NHCOR₀, —COR_{D}, —COOR₀, —CON(R₀)(R₁), wherein R₀ and R₁ are the same or different, and are H, C₁-C₁₀ alkyl or aryl.

2. A compound of formula (I) according to claim 1, wherein
Y is a bivalent radical having the following meanings:
R′ and R″ are independently H or straight or branched C₁-C₆ alkyl;
a) straight or branched C₁-C₁₀ alkylene;
b) 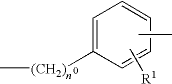
wherein R¹ is H, —COON or —OH, n⁰ is an integer from 0 to 5;
wherein the X moiety is not linked to —(CH₂)n⁰;
d) 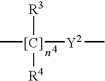
wherein:
n⁴ is an integer from 0 to 5;
R³ and R⁴ are H;
wherein the X moiety is linked to Y2;
Y² is an heterocyclic selected from the group consisting of: 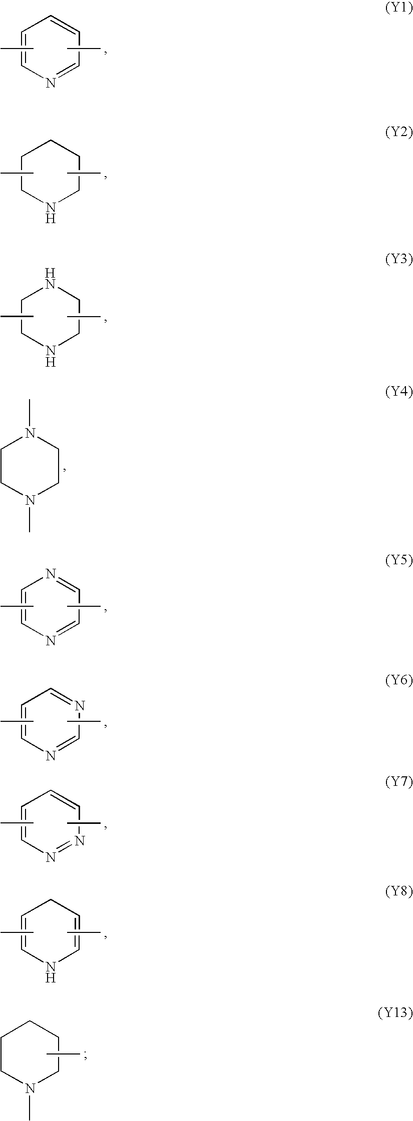
X is a moiety selected from the group consisting of: C₁-C₁₀ alkylene, —O—C₁-C₁₀ alkylene, —S—C₁-C₁₀ alkylene, —S(O)—C₁-C₁₀ alkylene and —S(O)₂—C₁-C₁₀ alkylene;
D has the same meanings according to claim 1.

3. A compound of formula (I) according to claim 2 selected from the group consisting of:

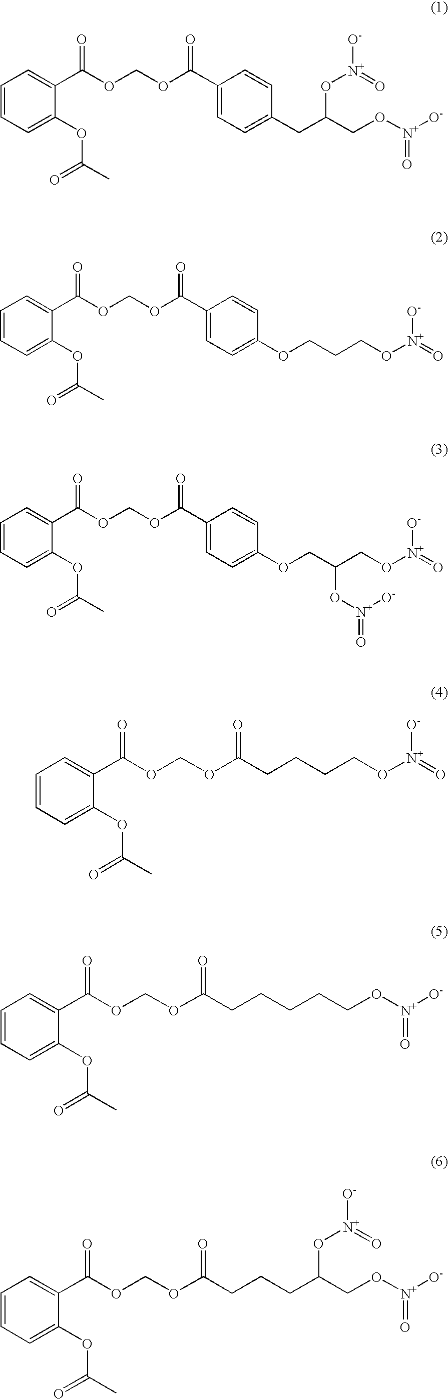

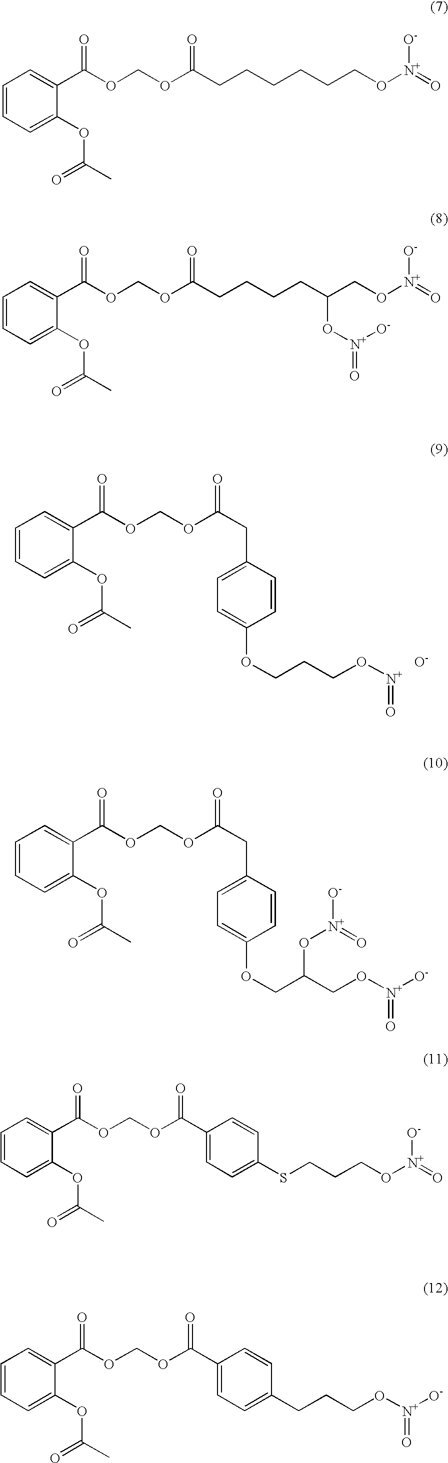

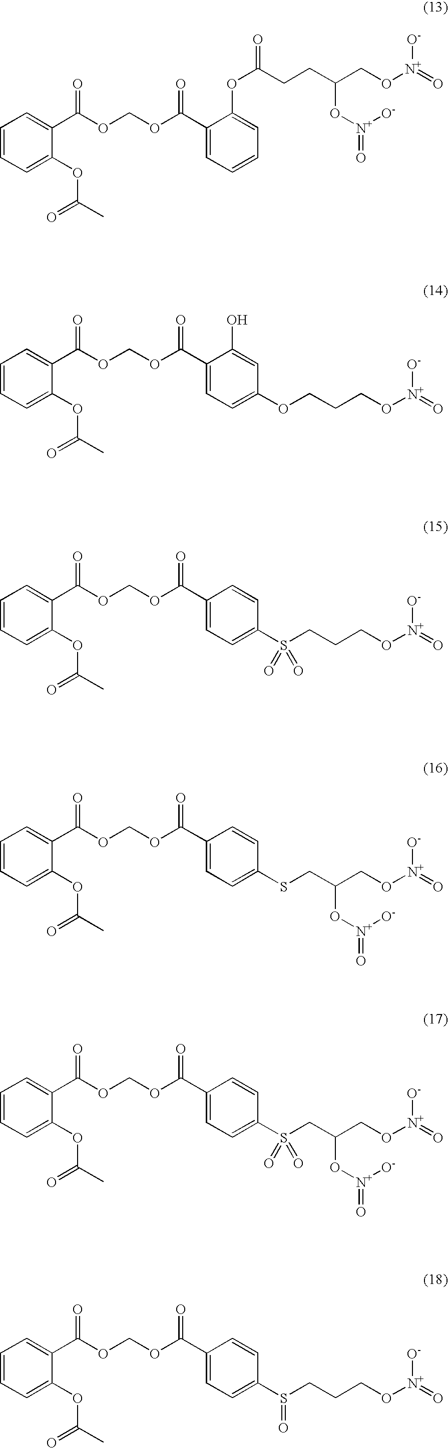

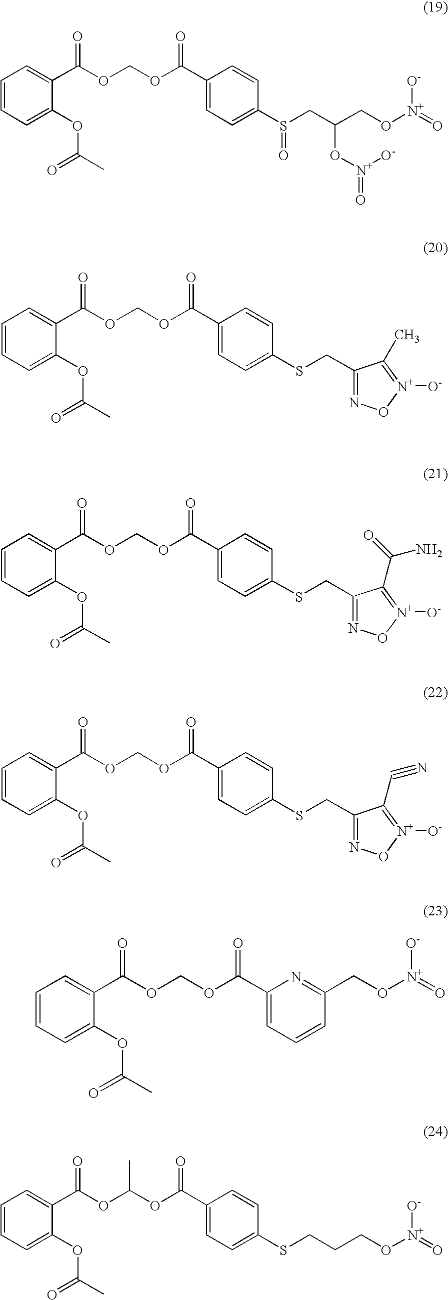

4. A compound of general formula (I) according to claim 1 for use as a medicament.

5. Use of a compound according to claim 1 for the preparation of an medicament having anti-inflammatory, antithrombotic and antiplatelet activity.

6. Use of a compound according to claim 1 for the preparation of an medicament for treating inflammation, pain, fever and cardiovascular diseases.

7. Use of a compound according to claim 1 for the preparation of an medicament for preventing or treating cancer diseases.

8. Use of a compound according to claim 7 for the preparation of an medicament for treating colon cancer, bladder cancer, prostate cancer.

9. A pharmaceutical composition comprising a pharmaceutically acceptable carrier and a pharmaceutically effective amount of a compound of general formula (I) and/or a salt or stereoisomer thereof as defined in claim 1.
